SEQUENCE LISTING (35 sequences)

SEQ ID NO: 1 - protein, 13 aa, Artificial Sequence
  Synthetic Peptide
CADTGGLSGKLTF

SEQ ID NO: 2 - protein, 15 aa, Artificial Sequence
  Synthetic Peptide
CAAGNTGGLSGKLTF

SEQ ID NO: 3 - protein, 16 aa, Artificial Sequence
  Synthetic Peptide
CAARPNTGGLSGKLTF

SEQ ID NO: 4 - protein, 16 aa, Artificial Sequence
  Synthetic Peptide
CAASGNTGGLSGKLTF

SEQ ID NO: 5 - protein, 11 aa, Artificial Sequence
  Synthetic Peptide
CAAHDNYQLIW

SEQ ID NO: 6 - protein, 11 aa, Artificial Sequence
  Synthetic Peptide
CAAKDNYQLIW

SEQ ID NO: 7 - protein, 11 aa, Artificial Sequence
  Synthetic Peptide
CAARYNYQLIW

SEQ ID NO: 8 - protein, 11 aa, Artificial Sequence
  Synthetic Peptide
CAASDNYQLIW

SEQ ID NO: 9 - protein, 11 aa, Artificial Sequence
  Synthetic Peptide
CAEDDNYQLIW

SEQ ID NO: 10 - protein, 11 aa, Artificial Sequence
  Synthetic Peptide
CAPDDNYQLIW

SEQ ID NO: 11 - protein, 12 aa, Artificial Sequence
  Synthetic Peptide
CAAGGDNYQLIW

SEQ ID NO: 12 - protein, 12 aa, Artificial Sequence
  Synthetic Peptide
CAAKGGNYQLIW

SEQ ID NO: 13 - protein, 12 aa, Artificial Sequence
  Synthetic Peptide
CAALDDNYQLIW

SEQ ID NO: 14 - protein, 12 aa, Artificial Sequence
  Synthetic Peptide
CAAMDDNYQLIW

SEQ ID NO: 15 - protein, 12 aa, Artificial Sequence
  Synthetic Peptide
CAARKDNYQLIW

SEQ ID NO: 16 - protein, 12 aa, Artificial Sequence
  Synthetic Peptide
CAASDDNYQLIW

SEQ ID NO: 17 - protein, 12 aa, Artificial Sequence
  Synthetic Peptide
CAATDDNYQLIW

SEQ ID NO: 18 - protein, 12 aa, Artificial Sequence
  Synthetic Peptide
CAGEGDNYQLIW

SEQ ID NO: 19 - protein, 12 aa, Artificial Sequence
  Synthetic Peptide
CAVMDDNYQLIW

SEQ ID NO: 20 - protein, 12 aa, Artificial Sequence
  Synthetic Peptide
CAVSDDNYQLIW

SEQ ID NO: 21 - protein, 13 aa, Artificial Sequence
  Synthetic Peptide
CAASSDDNYQLIW

SEQ ID NO: 22 - protein, 13 aa, Artificial Sequence
  Synthetic Peptide
CAASTNDNYQLIW

SEQ ID NO: 23 - DNA, 21 bp, Artificial Sequence
  Synthetic Peptide
tgtgcagccatggatgacaac

SEQ ID NO: 24 - DNA, 21 bp, Artificial Sequence
  Synthetic Peptide
tgtgcagcaatggatgacaac

SEQ ID NO: 25 - DNA, 21 bp, Artificial Sequence
  Synthetic Peptide
tgtgcagccatggatgacaac

SEQ ID NO: 26 - DNA, 21 bp, Artificial Sequence
  Synthetic Peptide
tgtgcagcaatggatgacaac

SEQ ID NO: 27 - DNA, 21 bp, Artificial Sequence
  Synthetic Peptide
tgtgcagcgatggatgacaac

SEQ ID NO: 28 - DNA, 21 bp, Artificial Sequence
  Synthetic Peptide
tgtgcagcaaggccgaatact

SEQ ID NO: 29 - DNA, 21 bp, Artificial Sequence
  Synthetic Peptide
tgtgcagcccgaccgaatact

SEQ ID NO: 30 - DNA, 21 bp, Artificial Sequence
  Synthetic Peptide
tgtgcagcaagaaaggacaac

SEQ ID NO: 31 - DNA, 21 bp, Artificial Sequence
  Synthetic Peptide
tgtgcagcaagaaaagacaac

SEQ ID NO: 32 - DNA, 21 bp, Artificial Sequence
  Synthetic Peptide
tgtgcagcaaggaaggacaac

SEQ ID NO: 33 - DNA, 21 bp, Artificial Sequence
  Synthetic Peptide
tgtgcagcaaggaaagacaac

SEQ ID NO: 34 - DNA, 21 bp, Artificial Sequence
  Synthetic Peptide
tgtgcagcccatgacaactat

SEQ ID NO: 35 - DNA, 21 bp, Artificial Sequence
  Synthetic Peptide
tgtgcagcacatgacaactat

(19) United States
(12) Patent Application Publication
(10) Pub. No.: US 2011/0044939 A1
(43) Pub. Date: Feb. 24, 2011

(54) REGULATORY T CELLS IN ADIPOSE TISSUE

(75) Inventors: Markus Feuerer, Brookline, MA (US); Diane J. Mathis, Brookline, MA (US); Steven Shoelson, Natick, MA (US); Christophe O. Benoist, Brookline, MA (US)

Correspondence Address: FISH & RICHARDSON P.C. (BO), P.O. BOX 1022, MINNEAPOLIS, MN, 55440-1022, US

(73) Assignee: JOSLIN DIABETES CENTER, INC., Boston, MA (US)

(21) Appl. No.: 12/666,563

(22) PCT Filed: Jun. 27, 2008
(86) PCT No.: PCT/US08/68658

§ 371 Date: Nov. 12, 2010

Related U.S. Application Data
(60) Provisional application No. 60/937,449, filed on Jun. 27, 2007.

(51) Int. Cl. A61K 38/20; A61K 35/14; A61P 3/04; C12N 5/0783; A61P 5/50

(52) U.S. Cl. 424/85.2; 424/93.71; 435/372.3

(57) ABSTRACT

Methods of preventing, delaying, or reducing the development or severity of obesity-associated disorders, including administering Fat-specific regulatory T cells, or administering factors secreted by said T cells.

### CLAIM OF PRIORITY

This application claims the benefit of U.S. Provisional Patent Application Ser. No. 60/937,449, filed on Jun. 27, 2007, the entire contents of which are hereby incorporated by reference.

### FEDERALLY SPONSORED RESEARCH OR DEVELOPMENT

The inventions described herein were made with Government support under Grant No. AI51530, DK51729 and DK73547, awarded by the National Institutes of Health, and Grant No. 2 P30 DK36836-20 from the National Institutes of Diabetes/Digestive/Kidney Diseases (NIDDK) to the Joslin Diabetes Center's Diabetes and Endocrinology Research Center (DERC) core facilities. The Government has certain rights in the invention.

### TECHNICAL FIELD

This invention relates to methods of reducing inflammation in adipose tissue.

### BACKGROUND

Chronic, low-grade inflammation, in particular of adipose tissue, is a critical element in obesity and its co-morbidities, insulin resistance and type-2 diabetes (Tilg and Moschen, Nat. Rev. Immunol. 6, 772-783 (2006); Shoelson and Goldfine, J Clin Invest 116, 1793-1801 (2006)).

### SUMMARY

As described herein, FoxP3⁺CD25⁺CD4⁺ regulatory T (Treg) cells are readily detectable in the abdominal adipose tissue of normal adult mice, accumulating with age to the unusually high fraction of around 50% of CD4⁺ T lymphocytes. According to a number of criteria, these abdominal fat Treg cells have a unique phenotype, distinct from that of previously described regulatory T cell populations. Treg cells are drastically reduced in the abdominal fat of insulin-resistant mouse models of obesity, but not in subcutaneous fat, nor elsewhere. Abdominal fat Treg cells express high levels of the anti-inflammatory cytokine IL-10, which directly reduces adipocyte secretion of inflammatory mediators. FOXP3 transcripts are found at higher levels in subcutaneous than omental fat of obese individuals. This population of specialized Treg cells in adipose tissue controls the activities of non-immune neighboring cells in potentially pathological contexts; thus, these cells and their anti-inflammatory properties can be used to inhibit elements of the metabolic syndrome.

In one aspect, the invention provides methods for inhibiting, preventing, delaying, or reducing the development or severity of obesity-associated disorders in a subject. The methods include obtaining an initial population of Foxp3+CD25+CD4+ regulatory T cells from a first subject; culturing said initial population of T cells, optionally in the presence of IL-10 and/or adiponectin, until said initial population has increased in size (i.e., in number of cells) to a predetermined level to form an increased population, and selecting cells that express one or more of interleukin (IL)-10, Gm1960, chemokine (C—C motif) receptor 1 (CCR1), CCR2, CCR9, chemokine (C—C motif) ligand 6 (CCL6), chemokine (C—X—C motif) ligand 5 (CXCL5), CXCL7, CXCL10, CXCL2, integrin alpha V, and activated leukocyte cell adhesion molecule (Alcam), thereby forming a population of fat-tissue specific regulatory T cells; and administering said population of fat-tissue specific regulatory. T cells to a recipient, e.g., the first (same) or a second (different) subject.

In another aspect, the invention provides methods for producing a population of fat-tissue specific regulatory T cells. The methods include obtaining an initial population of Foxp3+CD25+CD4+regulatory T cells; culturing said initial population of T cells, optionally in the presence of IL-10 and/or adiponectin, and selecting cells from the cultured initial population of T cells that express one or more of IL-10, Gm1960, CCR1, CCR2, CCR9, CCL6, CXCL5, CXCL7, CXCL10, CXCL2, integrin alpha V, and Alcam, thereby forming a population of fat-tissue specific regulatory T cells. In some embodiments, the methods further include administering said population of fat-tissue specific regulatory T cells to a recipient, e.g., the same or a different subject.

In a further aspect, the invention provides methods for producing a population of fat-tissue specific regulatory T cells. The methods include obtaining an initial population of Foxp3+CD25+CD4+ regulatory T cells; engineering said initial population of T cells to express IL-10 and optionally culturing said cells in the presence of adiponectin; and culturing said cells until the cells (i) secrete IL-10 and (ii) express one or more of Gm1960, CCR1, CCR2, CCR9, CCL6, CXCL5, CXCL7, CXCL10, CXCL2, integrin alpha V, and Alcam, and selecting said cells from the population of engineered, cultured cells, thereby forming a population of fat-tissue specific regulatory T cells. In some embodiments, the methods further include administering said population of fat-tissue specific regulatory T cells to a recipient, e.g., the same or a different subject.

In some embodiments, the initial population of cells comprises regulatory T cells from peripheral blood. In some embodiments, the initial population of cells comprises regulatory T cells from a fat tissue of the first subject.

In some embodiments, said population of fat-tissue specific regulatory T cells is administered to the recipient systemically. In some embodiments, said population of fat-tissue specific regulatory T cells is administered locally to a fat tissue of the recipient.

In some embodiments, the population of fat-tissue specific regulatory T cells express all of Gm1960, CCR1, CCR2, CCR9, CCL6, CXCL5, CXCL7, CXCL10, CXCL2, integrin alpha V, and Alcam. In some embodiments, the cells are engineered to express Fat Treg-specific TCRs, as described herein.

In some embodiments, the initial population of T cells is cultured in the presence of one or both of interleukin 2 (IL-2) and transforming growth factor beta (TGFβ).

In some embodiments, the initial population of cells is cultured in the presence of an anti CD3 antibody, and optionally a costimulatory molecule, e.g., an anti-CD28 antibody.

In another aspect, the invention provides populations of cells produced by a method described herein.

In yet an additional aspect, the invention provides methods for treating obesity and/or obesity-associated conditions, e.g., insulin resistance, metabolic syndrome, or type 2 diabetes, in a subject; the methods include administering a therapeutically effective amount of IL-10 and optionally adiponectin to the subject. In some embodiments, the IL-10 and adiponectin are administered systemically. In some embodiments, the IL-10 and adiponectin are administered locally to a fat tissue of the subject. In some embodiments, the IL-10 and adiponectin are administered in a single composition.

In another aspect, the invention provides methods for treating obesity and/or obesity-associated conditions, e.g., insulin resistance, metabolic syndrome, or type 2 diabetes, in a subject, the method comprising selecting a subject based on a diagnosis of overweight or obesity (e.g., a BMI of 25-29.9, or above 30), and administering a therapeutically effective amount of a composition comprising an interleukin (IL)-2:anti-IL-2 monoclonal antibody (mAb) complex to the subject. In some embodiments, the subject does not have an autoimmune disorder (e.g., type 1 (autoimmune) diabetes); the methods can include selecting the subject on the basis that they do not have an autoimmune disorder.

Also provided herein are pharmaceutical compositions including IL-10 and adiponectin as active ingredients, and a physiologically acceptable carrier.

A “recipient” is a subject into whom a cell, tissue, or organ graft is to be transplanted, is being transplanted, or has been transplanted. An “allogeneic” cell is obtained from a different individual of the same species as the recipient and expresses “alloantigens,” which differ from antigens expressed by cells of the recipient. A “xenogeneic” cell is obtained from a different species than the recipient and expresses “xenoantigens,” which differ from antigens expressed by cells of the recipient.

A “donor” is a subject from whom a cell, tissue, or organ graft has been, is being, or will be taken. “Donor antigens” are antigens expressed by the stem cells, tissue, or organ graft to be transplanted into the recipient. “Third party antigens” are antigens that differ from both antigens expressed by cells of the recipient, and antigens expressed by the donor cells, tissue, or organ graft to be transplanted into the recipient. The donor and/or third party antigens may be alloantigens or xenoantigens, depending upon the source of the graft. An allogeneic or xenogeneic cell administered to a recipient can express donor antigens, i.e., some or all of the same antigens present on the donor stem cells, tissue, or organ to be transplanted, or third party antigens. Allogeneic or xenogeneic cells can be obtained, e.g., from the donor of the cells, tissue, or organ graft, from one or more sources having common antigenic determinants with the donor, or from a third party having no or few antigenic determinants in common with the donor.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a set of six graphs showing the results of cell sorting experiments. Upper row: T cell distribution in SVF fraction from abdominal fat tissue. Numbers on top indicate mean and SD for cells in lymphocyte gate after fixing and permeabilization, fraction of CD3⁺ T cells among lymphocyte gated cells and distribution of CD4⁺ and CD8⁺ T cells. Lower row: Percentage of Foxp3⁺CD25⁺ T cells in abdominal fat tissue gated on CD4⁺ or CD8⁺ T cells. Organs of 5 mice were pooled. Representative dot plots are shown.

FIG. 1B is a bar graph showing the frequency of Foxp3⁺CD4⁺ T cells in different organs. Mean and SD from at least three independent experiments are shown, whereas organs from 4-5 mice per experiment were pooled.

FIG. 1C is a line graph showing the kinetics of Treg cell appearance in abdominal and s.c. fat tissue as well as spleen.

FIG. 1D is a set of six photomicrographs showing the results of immunohistology of abdominal adipose tissue. Arrow heads indicate Foxp3 staining. Foxp3 expression is restricted to the nucleus. * refers to dead-adipocyte residue surrounded by a crown like structure formed by immune cells. 1D-iv shows control staining with isotype antibody. Original magnification: (i) 400×, (ii-vi) 1000×.

FIG. 2A is a line graph showing the results of a standard in vitro suppression assay. Spleen-derived CD4⁺ effector T cells (responder cells) were incubated at various ratios with different T cell populations.

FIGS. 2B-G are scatter graphs showing the results of analysis with Affymetrix M430v2.0 chips. Normalized expression values for the profiles of: Expression profiles directly comparing Treg cells between: (2B) fat vs. spleen, (2C) fat vs. LN, (2D) LN vs. spleen. Expression profiles directly comparing Tconv between: (2E) fat vs. spleen, (2F) fat vs. LN, (2G) LN vs. spleen. (2B-G) Numbers are calculated based on a cut-off of 2-fold from the individual comparisons. (2H-J)

FIGS. 2H-J are “Volcano” plots of gene expression data comparing p-values vs. fold change for probes from the consensus Treg signature (Fontenot et al., Immunity 22, 329-341 (2005); Hill et al., Immunity 25, 693-695 (2007)). Plotted for: (2H) spleen Treg vs. Tconv; (2I) fat Treg vs. Tconv; (2J) fat Treg vs. LN Tconv. Genes uniquely up or down regulated in fat Treg cells are highlighted in light grey and dark grey, respectively

FIGS. 2K and 2L are fold-change to fold change plots comparing Treg expression profiles between: (2K) fat Treg x-axis) and LN Treg (y-axis); (2L) spleen Treg x-axis) and LN Treg (y-axis). Genes uniquely up or down regulated in fat Treg cells are highlighted in light grey and dark grey, respectively.

FIG. 3A is a set of three bar graphs and a scatter plot showing relative RNA expression of selected genes from Treg and Tconv cells from LN and fat.

FIG. 3B is a set of eight scatter graphs showing the results of cell sorting experiments in which cells were isolated from the abdomen, spleen, lung, and liver of retired breeder B6 mice and the SVF fraction was stained for Foxp3, CD3, CD4, CD8, CD25 and CD103 and CTLA-4, gated on CD3/CD4 expression.

FIG. 3C is a set of three bar graphs showing relative RNA expression of IL-10, IFN-γ, and Tbet in Treg and Tconv cells from LN and fat.

FIGS. 3D and 3E are scatter plots of cytokine expression profiles from Treg and Tconv cells from spleen, lung and fat tissue. Shown are the profiles for IL-10, IFN-γ and IL-4, as well as TNFα in abdominal fat (3E). Representative dot plots of at least three independent experiments arc shown. Organs from 4-6 mice were pooled per experiment.

FIG. 4A shows results from gene analysis of abdominal fat and LN Treg and Tconv cells isolated from old male animals from the Limited (LTD) mouse line. The frequency of the CDR3α sequences was analyzed on a single cell base. Upper panel: graphic display of the TCR sequence in a heat map format from Treg and Tconv cells. Second panel: Percentage of popular sequences as defined by >2 in the fat or >2 in the LN are shown for thymus, LN and fat. Third and fourth panels: Nucleotide sequences of fat (third) and conventional (fourth panel) Treg cell TCR sequences that showed multiple nucleotide sequence.

FIG. 4B is a set of eight scatter graphs of results of cells sorting of cells isolated from abdominal adipose tissue, LN, liver and lung from retired breeder B6 mice. The SVF fraction was stained for Foxp3, CD3, CD4, CD8 and the activation marker CD69 and Ly6c. Representative dot plots are shown.

FIGS. 5A-5I show results in three mouse models of obesity: ob/ob, agouti and high fat diet. (5A-C) Abdominal adipose tissue from ob/ob and heterozygote ob/wt mice was analyzed for Treg cell frequency. (5A) representative dot plots of 13-week-old ob/wt and ob/ob mice. (5B) bar graph showing the total number of Treg cells per one gram fat. (5C) line graph showing the changes of Treg representation over age. Mean and SD are shown. (5D) bar graph showing the percentage of Treg cells in abdominal adipose tissue of 24-week-old agouti (ag/wt) or littermate (wt) mice. (5E) bar graph showing the percentage of fat Treg cells in mice fed for 29 weeks with high fat diet (HFD) and normal chow (NC). (5F) dot plot showing the correlation of HOMAR-IR and fraction of Treg cells. (5G-I) bar graphs showing the observed changes of Treg cell proportion in adipose tissue of the three obesity models were not reflected in other organs. (G) ob/ob, (H) agouti, (I) HFD.

FIGS. 6A-i to 6A-vii show the results of a loss-of-function experiment conducted by depleting Treg cells expressing DTR. 10-week-old male mice, either DTR-positive or -negative, were treated every other day for 4 days (6A-i-iii) or 9 days (6A-iv-vii) with DT. (6A-i) Scatter graph showing the percentage of Treg cells from spleen or the abdominal fat after 4 days of treatment. (6A-ii, iii) Bar graph and western blot showing that Treg depletion affects insulin signaling in epididymal WAT and liver. Immunoprecipitation and Western blotting of insulin IR shows a decrease in IR phosphorylation (pIR) in epi WAT and liver without differences in muscle and spleen. 6A-ii is a bar graph of the quantification of pIR normalized by total IR. (N≧4, *P<0.004, t test); (6A-iii) shows the blot data. (6A-iv) is a bar graph with an inset scatter graph, illustrating the percentage of Treg cells from the abdominal fat (upper panel) or spleen (lower panel) after 9 days of treatment, with a representative dot plot as an insert. (6A-v) is a pair of bar graphs; the upper panel shows RNA Expression of TNF-α, IL-6, A20, RANTES and SAA3 from abdominal adipose tissue. Three mice per group, one of two independent experiments is shown. The lower panel shows a comparison of RNA expression of RANTES and SAA3 in spleen, lung and abdominal fat (epi fat). (6A-vi and vii) Bar graphs of fasting insulin and glucose levels after 9 days of treatment. Six mice per group from two independent experiments were pooled. Significance was determined by Mann-Whitney U test.

FIG. 6B-i to vii show the results of a gain-of-function experiment, which included in situ expansion of Treg cells via injection of a monoclonal antibody specific for IL-2 coupled with recombinant IL-2. (6B-i and ii) Dot plots (6B-i) and summarizing bar graph (6B-ii) showing Treg cells from spleen and abdominal fat tissue (epi fat) from mice fed normal chow (NC) or with 15 weeks of high-fat diet (HFD). Treated with IL-2/anti-IL2 complex or saline for 6 days and analyzed on day 14 (n=6 for each group). Graphs are also presented showing fasting insulin (6B-iii), blood glucose (6B-iv) HOMA-1R (6B-v), and a GTT (6B-vi) of mice described in (6B-i and 6B-ii). (6B-vii) Bar graph showing the calculated area under the curve (AUC) from all mice tested by GTT(n=11), including the dataset described in (vi. p-values were calculated with T-test.

FIG. 7A is a sert of five bar graphs and a line graph. Left panel: IL-10 can reverse TNF-α mediated inflammatory changes in differentiated adipocytes. Expression of IL-6, MMP3, SAA3 and RANTES were measured with qPCR under unmanipulated culture conditions (control); adipocytes were treated with TNF-a (TNF); cells were treated with 1 ng/ml IL-10 (IL-10) alone; or cells were treated with TNF-α and IL-10 (TNF+IL-10). Middle panel: Relative expression of IL-6 in differentiated adipocytes, dose response curve of IL-10. TNF: TNF-α and different concentrations of IL-10. No TNF: only IL-10. Representative experiments of 2-4 are shown. Left panel: Expression of SAA3, RANTES, IL-6 and Glut4 in differentiated adipocytes un-manipulated (M) or treated with TNF-α, IFN-γ and IL-1β. Representative experiments of 2-4 are shown.

FIGS. 7B-i-Bii are line graphs of expression of FOXP3, CD3 and CD69 was measured by quantitative PCR in paired human omental and s.c. adipose samples from mostly obese individuals (BMI range: 25.5-56.43, average: 44.85). Plotted are the ratios of FOXP3 vs. CD3 for omental and s.c. adipose tissue (7B-i) and for CD3 vs. CD69 (7B-ii). 13 individual donors are shown.

FIG. 8 is a comparison of fat Treg-cell-specific genes with genes specific for activated Treg cells. Top 50 genes from the ratio: fat Treg cells vs. LN Treg cells and top 50 genes from the ratio: in vitro activated Treg cells vs. ex vivo Treg cells (both spleen, and day 4 after CD3/CD28 activation plus 2000 U IL-2). Expression values were row normalized and shown for individual replicates from different Treg cell populations (fat Treg cells, LN Treg cells, spleen Treg cells and activated Treg cells).

FIG. 9 is a list of fat Treg-specific genes. The fat Treg unique signature included genes specifically over- or under-represented in fat Treg cells and was generated by including genes 2-fold or more over- or under-expressed in fat Treg cells vs. fat Tconv cells as well as more then a 2-fold difference between fat Treg vs. LN Tconv cells. To exclude the classical Treg-specific genes, LN Treg vs. LN Tconv had to be less then 1.25 fold for over- or more then 0.8 for under-represented genes. Shown are the ratios for the 629 fat Treg-specific genes for fat Treg vs. fat Tconv, LN Treg vs. LN Tconv and spleen Treg vs. spleen Tconv.

FIGS. 10A-B show the top 145 genes (10A) and bottom 135 genes (10B) over- and under-expressed in fat Treg vs. fat Tconv cells. Expression values were row-normalized and presented in alphabetic order for Treg and Tconv cells from different organs (spleen, LN, thymus, and abdominal fat).

### DETAILED DESCRIPTION

The present invention is based, at least in part, on the discovery of a unique population of regulatory (Treg) T cells in fat tissues. These cells are characterized by the expression of a unique set of genes, including the overexpression of interleukin (IL)-10, when compared with lymph node (LN) Tregs.

The methods described herein take advantage of the properties of these cells by providing methods in which populations of these cells are transplanted into obese or pre-obese subjects, or in which factors secreted by these cells are administered to obese or pre-obese subjects. Pre-obese subjects are subjects who are at risk of developing obesity, i.e., have one or more risk factors for obesity, including but not limited to: high risk lifestyle factors (e.g., inactivity/sedentariness, age, psychological factors, consumption of a high fat diet, consumption of excessive calories, consumption of alcohol, certain medications, and cigarette smoking), genetics, and the presence of overweight BMI 25-29.9. In some embodiments, the subjects are selected on the basis that they are overweight or obese. In some embodiments, the subjects are selected on the basis that they do not have an autoimmune disease. In some embodiments, the subjects are insulin resistant.

T regulatory (Treg) cells

Treg cells are a lineage of CD4+ T lymphocytes specialized in controlling autoimmunity, allergy and infection (Sakaguchi,S. et al. Immunol Rev. 212, 8-27 (2006); Fontenot and Rudensky, Nat. Immunol 6, 331-337 (2005)). Initially characterized by surface-display of the interleukin(IL)-2 receptor α chain, CD25, and later by expression of the transcription factor FoxP3, naturally occurring Treg cells normally constitute about 10-20% of the CD4+T lymphocyte compartment. Typically, they regulate the activities of T cell populations, but they can also influence certain innate immune system cell types (Maloy et al., J. Exp. Med. 197:111-119 (2003); Murphy et al., J. Immunol. 174:2957-2963 (2005); Nguyen et al., Arthritis Rheum. 56, 509-520 (2007)).

As described herein, a population of special Tregs exists in higher numbers in fat tissues of normal weight individuals, but lower numbers in fat of overweight (BMI 25-29.9) and obese individuals (BMI 30 and above). These cells, which are called “fat Tregs” herein, are believed to play a role in regulating fat tissues, and are expected to reduce the development or severity of obesity-associated disorders.

The methods described herein include ways to provide useful populations of these special fat Tregs, starting either from an initial population of cells that includes a smaller number of fat Tregs, or non-fat Tregs, e.g., Tregs obtained from peripheral blood or other tissues. This initial population can be obtained using methods known in the art, and should be designed for optimal purity and viability of the cells.

The methods can include treating this initial population of cells with a cocktail of factors that optionally include IL-10 and adiponectin, and optionally additional factors, e.g., chemokines, e.g., CCR1, CCR9, or AA467197, and/or growth factors, e.g., IL-6 or transforming growth factor beta (TGH-β), until said initial population has increased in size to a predetermined level, and the cells (i) secrete IL-10, i.e., at levels significantly higher than levels secreted by non-fat T-regs, and (ii) express one or more, e.g., two, three, four, five, six, seven, or all eight of Gm1960, CCR1, CCR2, CCR9, CCL6, CXCL5, CXCL7, CXCL10, CXCL2, integrin alpha V, and Alcam. These cells can be selected using methods known in the art.

The IL-10 and adiponectin and additional factors, can be obtained from a commercial source, or can be produced using standard protein production and purification methods, e.g., by expression in a cultured cell system and affinity purified.

In some embodiments, rather than culturing the cells in the presence of the proteins, the cells are engineered to express IL-10 and adiponectin, and optionally additional factors.

In general, the initial population of cells will be cultured in the presence of a T cell receptor ligand, e.g., anti-CD3 antibody, and optionally a costimulatory molecule, e.g., anti-CD28 antibody, to engage the T cell receptors and activate the cells to encourage proliferation. In some embodiments, the cells will be grown in the presence of one or more growth factors, e.g., IL-6 or transforming growth factor beta (TGH-β),

Methods for detecting gene expression are well known in the art, and include, e.g., PCR-based methods, chip-based methods, and hybridization based methods.

The sequences of the mRNAs for IL-10, adiponectin, Gm1960, CCR1, CCR2, CCR9, CCL6, CXCL5, CXCL7 CXCL10, CXCL2, integrin alpha V, and Alum are available in public databases, e.g., as follows:

| Gene | GenBank ID |
|---|---|
| Homo sapiens interleukin 10 (IL10) | NM_000572.2 |
| Homo sapiens Adiponectin | NM_004797.2 |
| Mus musculus AA467197 | AA467197.1 |
| Homo sapiens Gm1960 | NC_000071.4 |
| Homo sapiens chemokine | NM_001295.2 |
| (C-C motif) receptor 1 (CCR1) | |
| Homo sapiens chemokine | NM_000647.4, |
| (C-C motif) receptor 2 (CCR2) | NM_000648.2 |
| Homo sapiens chemokine | NM_031200.1, |
| (C-C motif) receptor 9 (CCR9) | NM_006641.2 |
| Mus musculus chemokine (C-C motif) | NM_009139.3 |
| ligand 6 (CCL6) | |
| Homo sapiens chemokine (C-X-C motif) | NM_002994.3 |
| ligand 5 (CXCL5) | |
| Homo sapiens pro-platelet basic protein | NM_002704.2 |
| (chemokine (C-X-C motif) ligand 7) (CXCL7, | |
| PPBP) | |
| Homo sapiens chemokine (C-X-C motif) | NM_001565.1 |
| ligand 10 (CXCL10) | |
| Homo sapiens integrin alpha V | NM_002205.2, |
| | BC126231.1 |
| Homo sapiens activated leukocyte | NM_001627.2 |
| cell adhesion molecule (ALCAM) | |
| Homo sapiens chemokine (C-X-C motif) | NM_002089.3, |
| ligand 2 (CXCL2), | BC015753.1 |

In some embodiments, the methods described herein can include transfecting the initial population of cells with sequences encoding chemokines or chemokine receptors, e.g., AA467197, Gm1960, CCR1, CCR2, CCR9, CCL6, CXCL5, CXCL7 CXCL10, or CXCL2.

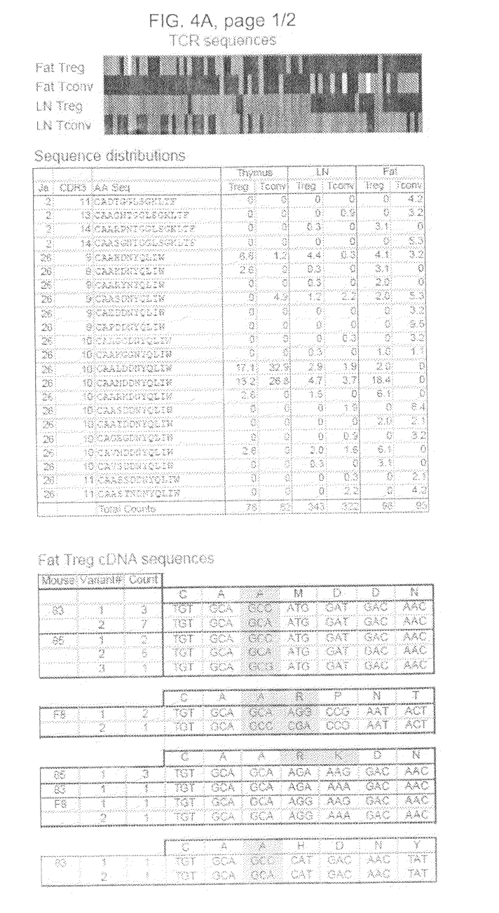

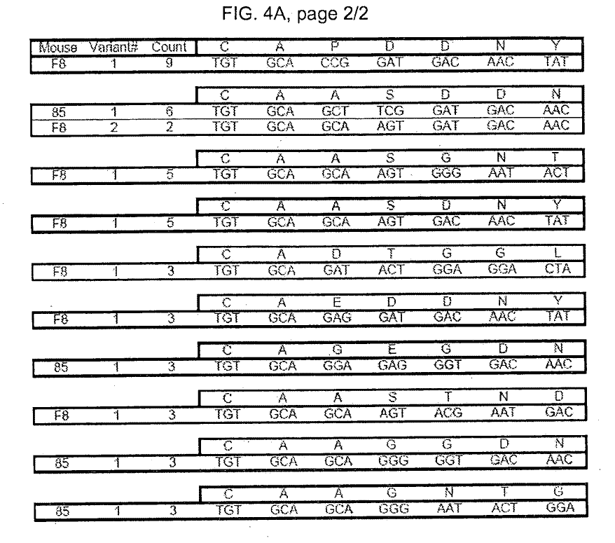

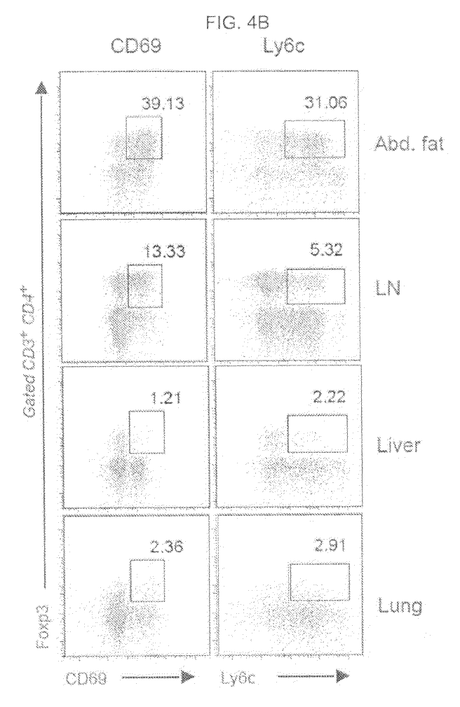

In some embodiments, the methods described herein can include transfecting the initial population of cells with sequences encoding Fat Treg-specific TCR sequences, e.g., as shown in FIG. 4, to encourage the cells to home to adipose tissues. Methods known in the art can be used to do this, e.g., transfecting the cells with one or more expression vectors encoding one or more TCRs.

The methods described herein can include the use of these sequences, or sequences that are substantially identical to these sequences. As used herein, “substantially identical” refers to a nucleotide sequence that contains a sufficient or minimum number of identical or equivalent nucleotides to the reference sequence, such that homologous recombination can occur. For example, nucleotide sequences that are at least about 80% identical to the reference sequence are defined herein as substantially identical. In some embodiments, the nucleotide sequences are about 85%, 90%, 95%, 99% or 100% identical.

To determine the percent identity of two nucleic acid sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 80% (in some embodiments, about 85%, 90%, 95%, or 100%) of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

Obesity-Associated Disorders

Obese individuals are at an increased risk of developing one or more serious medical conditions, which can cause poor health and premature death, as compared to their non-obese peers. Obesity is associated with an increased risk of numerous conditions, including Type 2 diabetes, insulin resistance, coronary heart disease, high blood pressure, cancer, carpal tunnel syndrome (CTS), chronic venous insufficiency (CVI), deep vein thrombosis (DVT), end stage renal disease (ESRD), gallbladder disease, impaired immune response, gout, and arthritis (i.e., rheumatoid arthritis (RA) and osteoarthritis (OA)), inter alia.

Methods of Treatment—Cell Therapy

In some embodiments, the methods described herein include the treatment of subjects who are, or who are likely to become, obese, by administration of a cell transplant comprising Fat Tregs, e.g., obtained by a method described herein. Methods of transplantation are known in the art, see, e.g., Kang et al., Am. J. Transplant. 7(6):1457-63 (2007).

Subjects who are the candidates for treatment using a method described herein include, inter alia, those who are obese (i.e., have a body mass index (BMI) of 30 or above), or are pre-obese (i.e., are likely to become obese). These subjects include individuals with a family history of obesity, a genetic or lifestyle predisposition to obesity, and/or a body mass index that indicates that they are overweight (i.e., BMI of 25-29.9)).

The Fat Tregs will generally be administered locally, i.e., into an area of the body characterized by the presence of fat tissues, e.g., omental or subcutaneous fat. In some embodiments, the Fat Tregs will be administered systemically, e.g., by intravenous administration.

As described herein, the Fat Tregs can be from the same person as they are intended to be transplanted to (i.e., autologous), or a different donor. The donor will generally be alive and viable, e.g., a volunteer donor. In some embodiments, more than one individual will donate the cells, e.g., the initial population of regulatory T cells will comprise cells from more than one donor.

In some embodiments, e.g., where the Tregs were not obtained from the recipient, the methods described herein can include the use of minimal myeloablative conditioning of the recipient. In some embodiments, minimal myeloablative conditioning can include the use, e.g., transitory use, of low doses of one or more chemotherapy agents, e.g., vincristine, actinomycin D, chlorambucil, vinblastine, procarbazine, prednisolone, cyclophosphamide, doxorubicin, vincristine, prednisolone, lomustine, and/or irradiating the thymus of the recipient mammal, e.g., human, with a low dose of radiation, e.g., less than a lethal dose of radiation plus chemotherapy agents. Lethal doses of conditioning include the administration of 14 Gy of irradiation plus cytarabine, cyclophosphamide, and methylprednisolone (Guinin et al, New Engl. J. Med., 340:1704-1714, 1999).

To prevent the development of graft-versus-host disease, additional treatment with a short course of methotrexate and cyclosporine starting on the day before transplantation using a bolus of 1.5 mg/kg over a period of 2-3 hours every 12 hours. This protocol should allow the reduction of irradiation conditioning to about 10 Gy or less, e.g., in some embodiments, about 5 Gy, about 2 Gy, about 1.5 Gy, about 1 Gy, about 0.5 Gy, about 0.25 Gy and the elimination of additional cytoreduction agents such as cytarabine, cyclophosphamide, and methylprednisolone treatments. Minimal myeloablative conditioning is typically achieved by administering chemical or radiation therapy at a level that will not destroy the recipient's immune function, and is similar to, or lower than, levels used for conventional cancer treatments, e.g., conventional chemotherapy.

Methods of Treatment—IL-10 and Adiponectin or IL-2:anti-IL-2 Monoclonal Antibody (mAb) Complex

In another aspect, the methods described herein include the treatment of subjects who are, or who are likely to become, obese, by administration of (i) IL-10, (ii) IL-10 plus adiponectin, or (iii) IL-2:anti-IL-2 monoclonal antibody (mAb) complex (Boyman et al., Expert Opin Biol Ther. 2006 December; 6(12):1323-31). Such administration can be systemic, or local, e.g., injection into an area of unwanted fat tissue, e.g., subcutaneous or omental fat. When both IL-10 and adiponectin are used, administration can be of a single composition, e.g., a pill or injectable solution, that includes both IL-10 and adiponectin, or can be administration of two separate compositions.

Dosage, toxicity and therapeutic efficacy of therapeutic compositions as described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. In general, when the IL-2:anti-IL-2 monoclonal antibody (mAb) complex is administered, a preferred dosage will be sufficient to increase numbers of Fat Tregs without increasing number of effector T cells.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the 1050 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Pharmaceutical Compositions

IL-10 and adiponectin, or an IL-2:anti-IL-2 monoclonal antibody (mAb) complex, as described herein can be incorporated into pharmaceutical compositions. Such compositions typically include the compounds (i.e., as active agents) and a pharmaceutically acceptable carrier. As used herein, “pharmaceutically acceptable carriers” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

For administration by inhalation, the compounds are typically delivered in the form of an aerosol spray from pressured container or dispenser which contains a suitable propellant, e.g., a gas such as carbon dioxide, or a nebulizer. Such methods include those described in U.S. Pat. No. 6,468,798.

The therapeutic compounds can also be prepared in the form of suppositories (e.g., with conventional suppository bases such as cocoa butter and other glycerides) or retention enemas for rectal delivery.

Therapeutic compounds comprising nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Example 1

### CD4+ T Cells in Adipose Tissue

Adipose tissue is composed of multiple cell types. Most prominent are adipocytes, but vascular endothelial cells, macrophages (Weisberg et al., J Clin Invest 112, 1796-1808 (2003); Xu et al., J Clin Invest 112, 1821-1830 (2003)) and lymphocytes (Caspar-Bauguil et al., FEBS.Lett. 579, 3487-3492 (2005); Wu et al., Circulation 115, 1029-1038 (2007)) are also found in the stromovascular fraction (SVF).

T cells were detected, quantified, and identified in adipose tissues from Male C57B1/6 (at different ages and retired breeders 25-35 weeks old), ob/ob and ob/wt mice, agouti mice, Foxp3GFP/B6 reporter mice 13, and Limited (LTD) mice bred in at the Joslin Diabetes Center or purchased from the Jackson Laboratory (Bar Harbor, Me.). Mice receiving a high fat diet (HFD) were fed for 29 weeks with a rodent diet of 45 kcal % fat from Research Diet (New Brunswick, N.J.; Cat# D12451). Abdominal (epidydimal) adipose tissue, s.c. adipose tissue, lung and liver were removed after flushing the organs through the portal vein and the right heart ventricle, cut into small pieces (or passed through a sieve in terms of liver) and digested for about 40 minutes with collagease type II (adipose tissue, Sigma) or collagenase type IV (Sigma). Cell suspensions were then filtered through a sieve (or, for the lung tissues, smashed through the sieve) and stromovascular fraction (SVF) was harvested after spinning. Cells were stained with: anti-CD4, anti-CD8, anti-CD3, anti-CD25 and anti-B220, anti-CD103, anti-GITR, anti-CD69, and anti-Ly6c antibodies, fixed and permeabilized according to the protocol (eBiosciences), followed by intracellular staining of Foxp3 (eBiosciences) and CTLA-4.

For intracellular cytokine staining, cells were stimulated with phorbol 12-myristate 13-acetate (PMA) (50 ng/ml) (Sigma) and ionomycin (1 nM) (Calbiochem) for 4 hours. GOLGISTOPT™ (BD Biosciences), a protein transport inhibitor containing monensin, was added to the culture at the recommended amount during the last three hours. Cells were stained with anti-CD4, anti-CD8, anti-CD3, anti-CD25 and anti-B220 antibodies and fixed and permeabilized according to the protocol (eBiosciences) followed by intracellular staining of Foxp3 (eBiosciences), 1IFN-gamma, TNF-alpha, IL-10, and/or IL-4. Cells were then analyzed using a MOFLO™ High-Performance Cell Sorter, COULTER® EPICS® XL™ or LSRII Flow Cytometer instruments, and FLOWJO cytometric data analysis and presentation software.

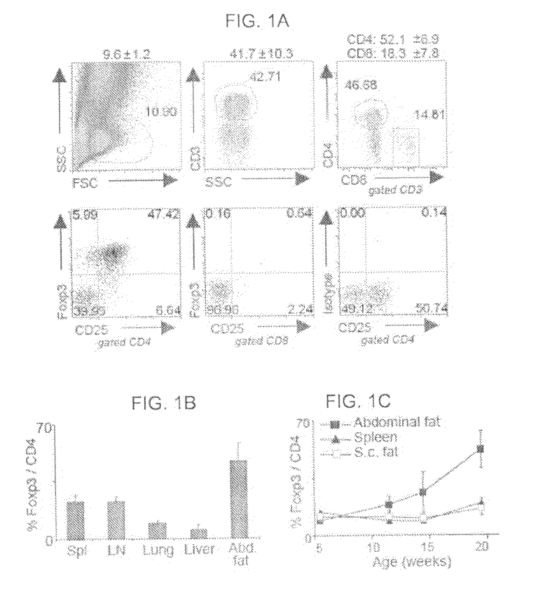

According to this multi-parameter flow cytometry, about 10% of SVF cells from the abdominal fat of 25-35-week-old C57B1/6 (B6) animals fell within the lymphocyte gate, close to half of which were of the CD3⁺ T lineage, split 2:1 between the CD4⁺ and CD8⁺ compartments, respectively (FIG. 1A, upper panels). Surprisingly, about half of the CD4⁺ T cells expressed Foxp3 and CD25 (FIG. 1A, lower panels), a much higher fraction than that normally found in lymphoid (e.g., spleen, lymph node (LN)) or non-lymphoid (lung, liver)) tissues (FIG. 1B), including in the subcutaneous fat (FIG. 1C). The two types of adipose tissue had similar, low levels of Treg cells at birth, with a progressive accumulation over time in the abdominal, though not subcutaneous, depot (FIG. 1C). About 15,000-20,000 Foxp3⁺ cells resided in one gram of epididymal adipose tissue.

Immunohistological examination was also performed. Abdominal (epidydimal) adipose tissue of 20-23 week old B6 mice was prepared and five-micron thick sections of formalin-fixed, paraffin-embedded adipose tissues were used for immunoperoxidase staining. After deparaffinization and rehydration, the peroxidase activity was blocked with 3% hydrogen peroxide in ethanol for 15 minutes. To retrieve antigen, the sections were treated in 10 mM citrate buffer (pH 6.0) using a digital decloaking chamber (Pacific Southwest Lab Equipment Inc., Vista, Calif.). The sections were then blocked with 1.5% rabbit serum for 15 minutes followed by incubation with 1:100 diluted monoclonal rat anti-mouse Foxp3 (clone FJK-16s, eBioscience, San Diego, Calif.) for an hour. VECTASTAIN ELITE ABC kit (Vector laboratories, Inc., Burlington, Calif.) was used to detect the primary antibody. The secondary antibody (rabbit anti-rat) was diluted 1:200 in 2% rabbit serum provided and applied to the sections for 30 minutes. The sections were then exposed to avidin-biotin complex for 30-40 minutes followed by 3,3′-diaminobenzidine (DAB) (DAKO, Carpinteria, Calif.) as substrate. The sections were counter-stained with Gill's Hematoxylin (Fisher Scientific, Pittsburgh, Pa.).

This immunohistological examination revealed Foxp3⁺ cells in the spaces between adipocytes, mainly, but not only, in regions where several adipocytes intersected (FIG. 1D, panels i-iii).

Fat tissue, especially from obese individuals, can host substantial numbers of macrophages, which accumulate in so-called “crown-like” structures, replete with dead-adipocyte residues (Weisberg et al., J Clin Invest 112, 1796-1808 (2003); Xu et al., J Clin Invest 112, 1821-1830 (2003); Cinti et al., J. Lipid.Res. 46, 2347-2355 (2005)). Treg cells were also observed in similar structures, in close proximity to macrophages and other leukocyte aggregates (FIG. 1D, panels iv and v). Given their known potency, this value very likely represents a biologically significant number—for example, transferring as few as 5,000-10,000 Tregs can protect a mouse from autoimmune diabetes (and many of the cells do not even survive the transfer process) (Herman et al., J. Exp. Med. 199:1479-1489 (2004); Chen et al., J. Immunol. 173:1399-1405 (2004)).

### Example 2

### Fat Treg Functional Profiling and Gene Expression Profiling

The present example describes experiments performed to determine whether the CD25⁺Foxp3⁺ cells in abdominal adipose tissue were of typical Treg phenotype.

First, a standard in vitro suppression assay was performed. Briefly, CD4⁺CD25⁺ Treg cells and CD4⁺CD25− conventional Tconv cells were sorted from adipose tissue and spleen from retired breeder mice. 2×10⁴CD4⁺CD25⁻ effector T cells from the spleen were cultured in 96-well plates in the presence of 0.5 mg/ml of anti-CD3 mAb (2C11) (BD Pharmingen, Inc., San Diego, Calif.) and T cell—depleted APCs. Treg and Tconv cells were titrated in at 1:1 to 1:4 ratios. Cultures were performed in triplicate, incubated for four days, and pulsed with ³H-thymidine for the last 16 hours of each experiment. Proliferation values were normalized to that of effector T cells alone.

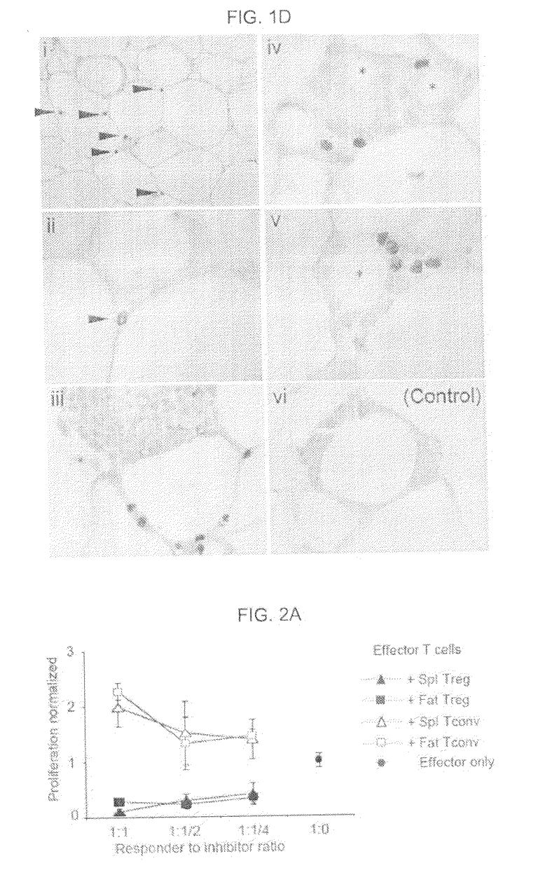

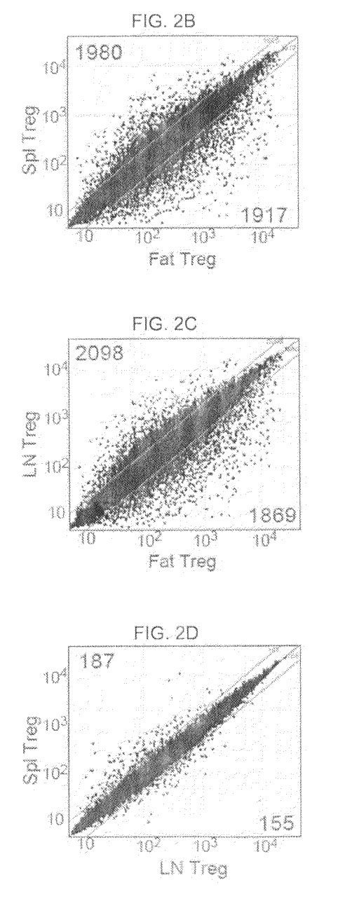

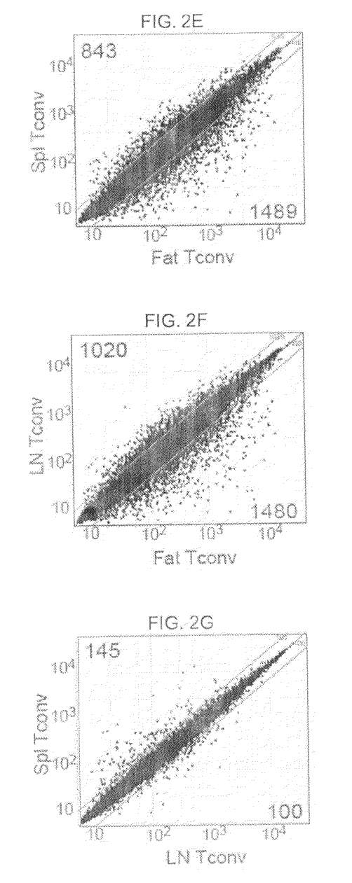

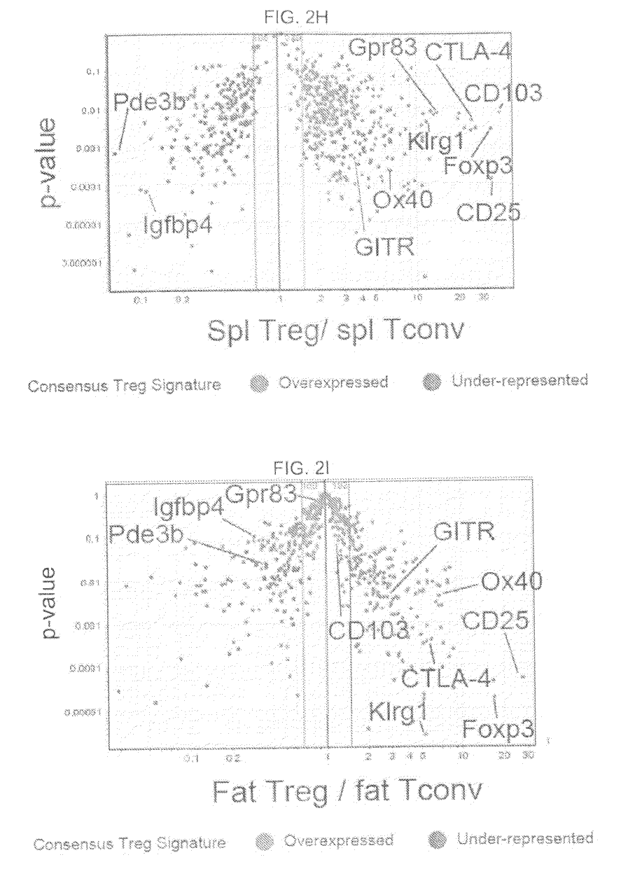

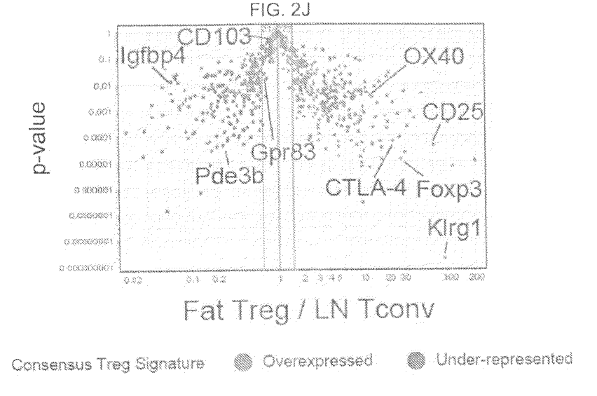

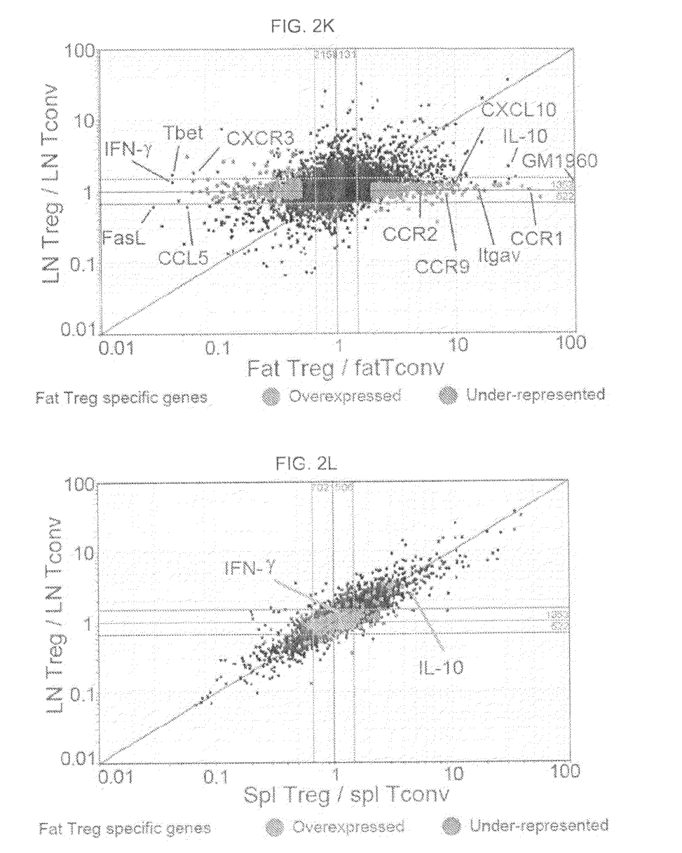

The fat Treg cells functioned as effectively as analogous cells isolated from the spleen in the standard in vitro assay (FIG. 2A). (Fat T conventional (Tconv) cells also performed as expected, i.e., there was no suppressive activity and a normal proliferative response (FIG. 2A)). However, the lability and low recoverable numbers of murine fat Tregs have so far made assaying their activities in in vivo suppressor assays technically difficult.

Next, the well-established transcriptional “Treg signature”, derived from the data of multiple groups (Fontenot et al., Immunity 22:329-341 (2005); Huehn et al., J. Exp. Med. 199:303-313 (2004); Herman et al., J. Exp. Med. 199:1479-1489 (2004); Hill et al., Immunity 25:693-695 (2007)), was evaluated as one indicator of function.

Lymph node and abdominal fat TCR⁺CD4⁺ and CD25^{hi} (Treg) or TCR⁺CD4⁺ and CD25⁻ (Tconv) cells were sorted from retired male breeder B6 mice and spleen Treg and Tconv cells were sorted from Foxp3^{GFP}/B6 reporter mice (Fontenot et al., Immunity 22, 329-341 (2005)). RNA was extracted with Trizol reagent and amplified for two rounds using the MessageAmp aRNA kit (Ambion), followed by biotin labeling using the BioArray High Yield RNA Transcription Labeling Kit (Enzo Diagnostics), and purified using the RNeasy Mini Kit (Qiagen). The resulting cRNAs (three independent datasets for each sample type) were hybridized to M430 2.0 chips (Affymetrix) according to the manufacturer's protocol. Initial reads were processed through Affymetrix software to obtain raw .cel files. Microarray data were background-corrected and normalized using the RMA algorithm implemented in the GenePattern software package (Reich et al., Nat.Genent. 38, 500-501 (2006)), and replicates averaged. A consensus Treg signature was compiled from four independent analyses (Fontenot et al., Immunity 22, 329-341 (2005); Hill et al., Immunity 25, 693-695 (2007)). The color-coding in the figures denoted genes 1.5 fold over- (light grey) or under- (dark grey) expressed in Tregs in all four reference datasets. The fat Treg-specific gene's set included loci specifically over- or underexpressed in fat Treg cells, and was generated by including genes 2-fold or more over- (light grey) or under-(dark grey) expressed in fat Treg vs. fat Tconv cells as well as more than 2-fold difference between fat Treg and LN Tconv cells. To exclude the classical Treg-specific genes, LN Treg vs. LN Tconv had to be less then 1.25 fold for over- or more then 0.8 for under-represented genes.

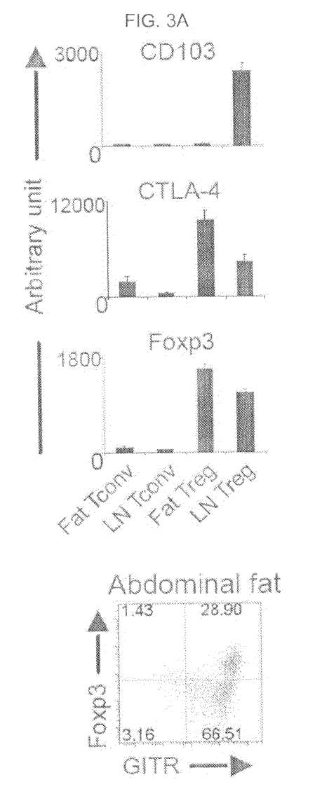

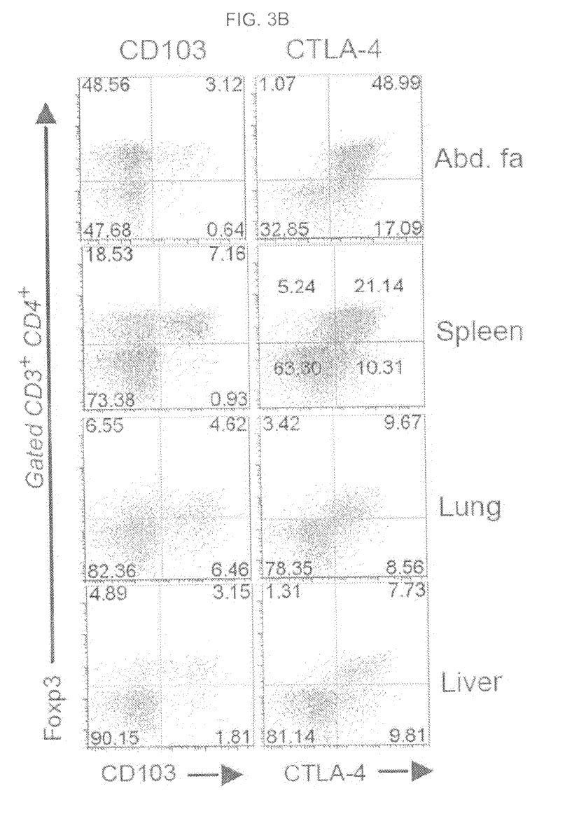

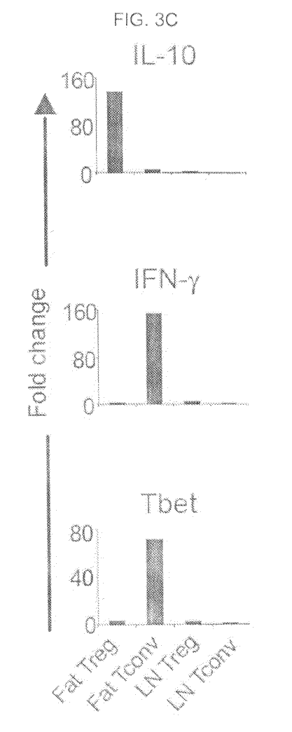

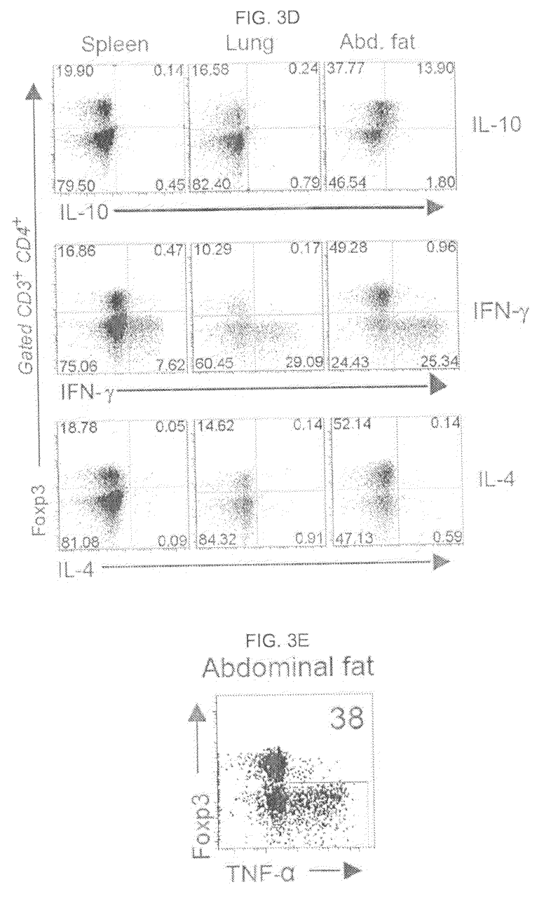

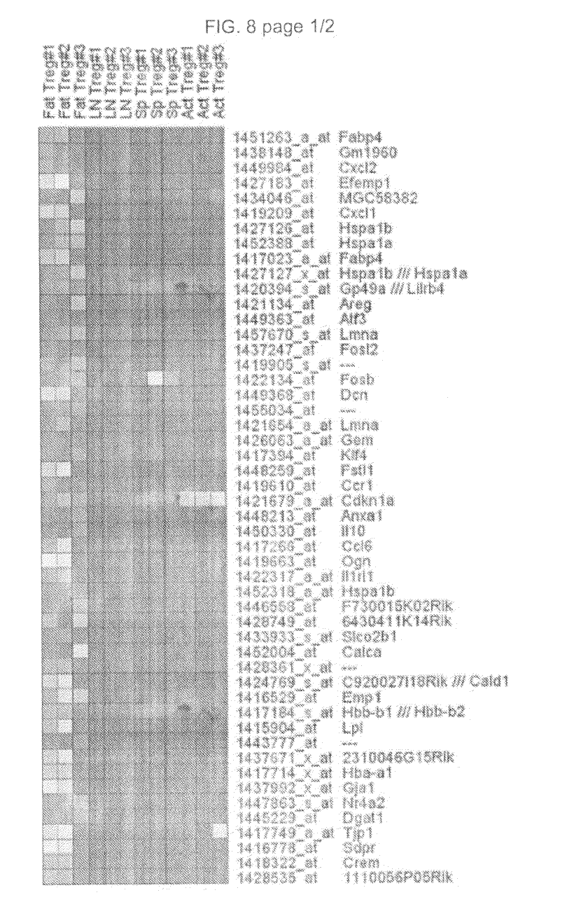

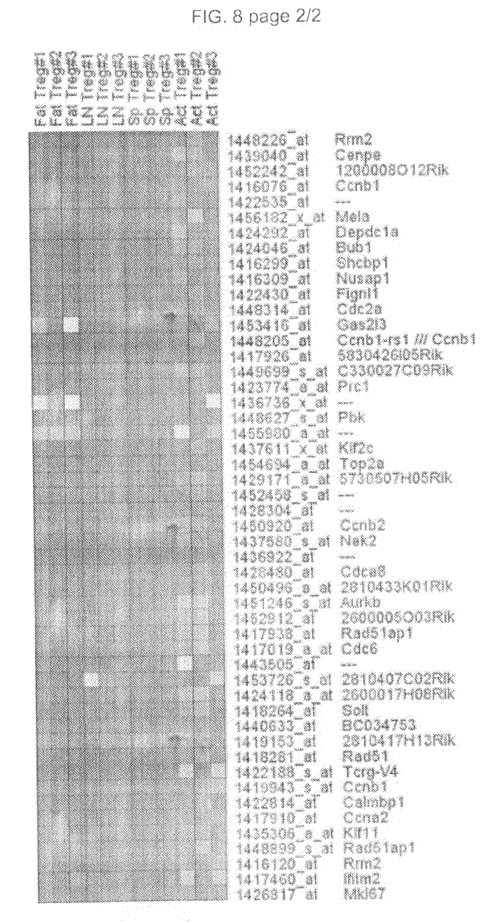

Clearly, the overall transcriptional profile of the Treg population from visceral fat differed from the patterns of its spleen and LN counterparts more than the latter two did from each other (FIG. 2, B-D). This observation also held for the Tconv populations at these sites, though not as strikingly so (FIG. 2, E-G). Focusing specifically on the documented Treg signature (Fontenot et al., Immunity 22:329-341 (2005); Huehn et al., J. Exp. Med. 199:303-313 (2004); Herman et al., J. Exp. Med. 199:1479-1489 (2004); Hill et al., Immunity 25:693-695 (2007)), the spleen data showed an excellent recapitulation of its major features; as anticipated, most genes known to be up-regulated in Tregs (light grey) descended to the right on the p-value vs fold-change (FC) “volcano” plot, while most down-regulated loci (dark grey) dropped to the left (FIG. 2H). Fully 93% of the signature was present. In contrast, evidenced by their position at the volcano summit, many of the signature Treg genes were not significantly up- or down-regulated in the corresponding population from visceral fat, e.g. CD103 and Gpr83 (FIG. 2, I and J). The data on CD103 (and others) were confirmed by flow cytometric analysis (FIG. 3B). These observations on the Treg signature were true whether the comparator was Tconv cells from the fat (FIG. 2I) or the LN (FIG. 2J), arguing that they reflect special features of adipose tissue Tregs. Nonetheless, fat-resident CD4⁺Foxp3⁺ cells were clearly Tregs, as much (63%) of the signature was intact, including over-expression of hallmark transcripts like those encoding CD25, GITR, CTLA-4, Ox40 and KIrgl, in addition to Foxp3 itself. Confirmation of the elevated expression of several of these signature genes in fat Tregs was obtained via RT-PCR and flow cytometric quantitation (FIG. 3, and data not shown). The gene-expression differences observed between Tregs isolated from the fat versus from the spleen and LN were not a simple reflection of different activation statuses, as a direct comparison between fat-derived and activated Tregs showed clearly divergent transcription patterns (FIG. 8).

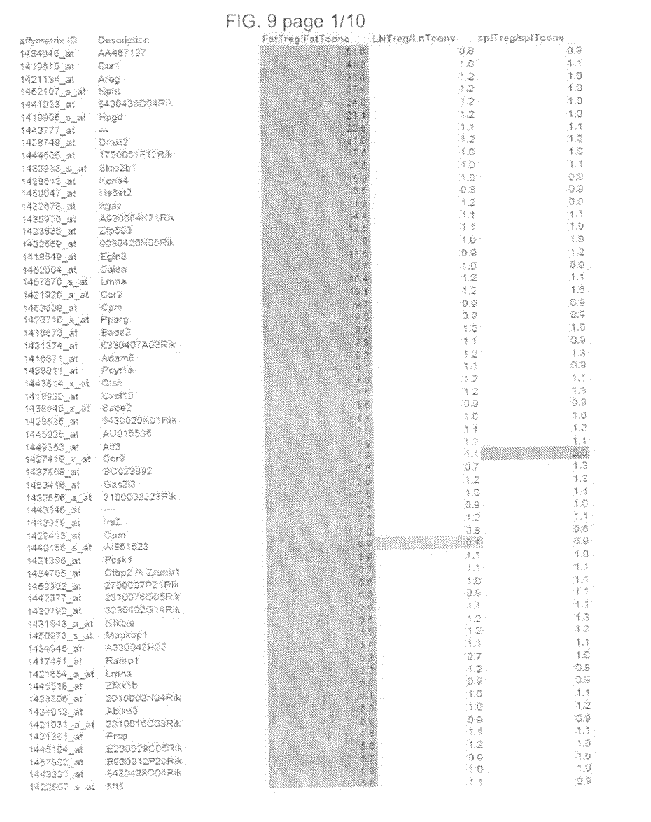

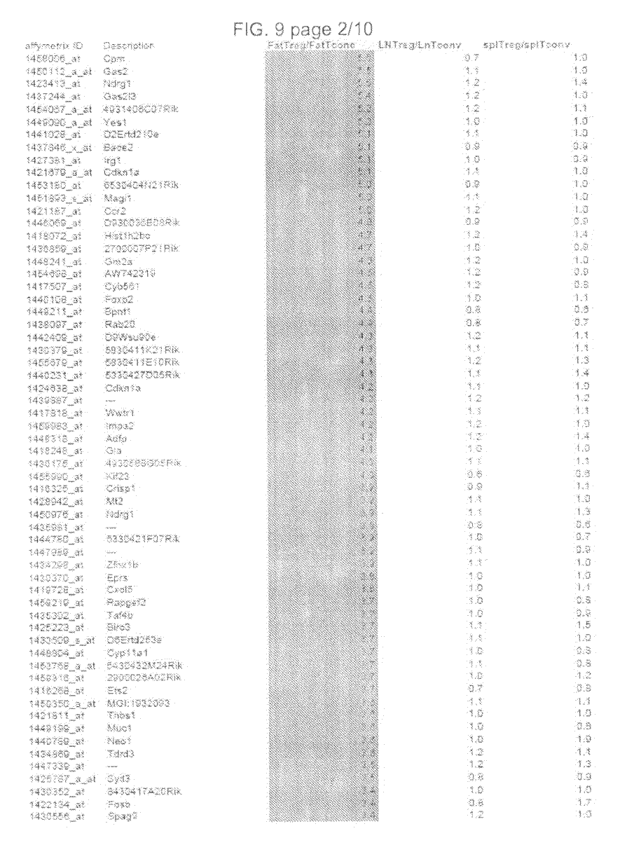

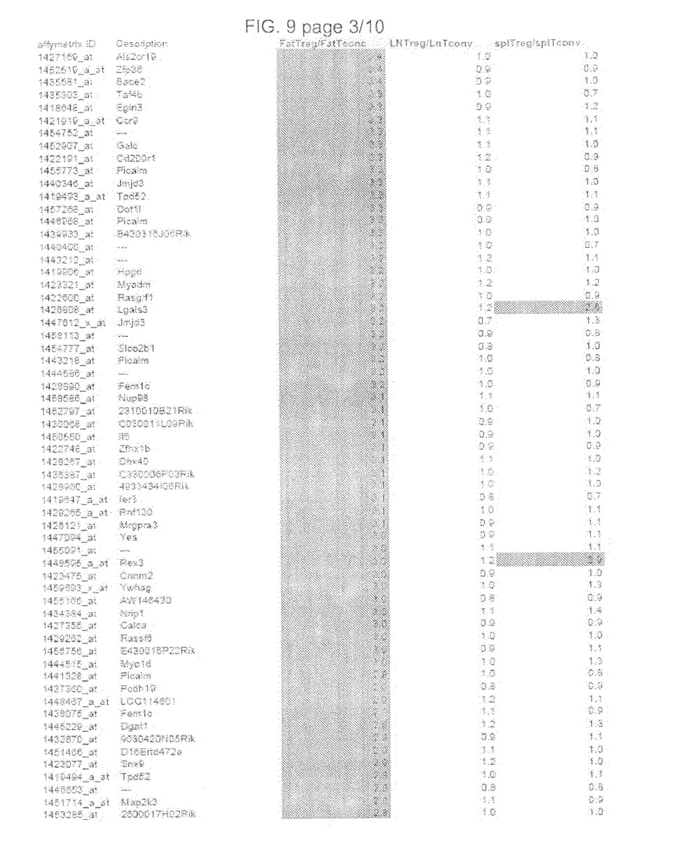

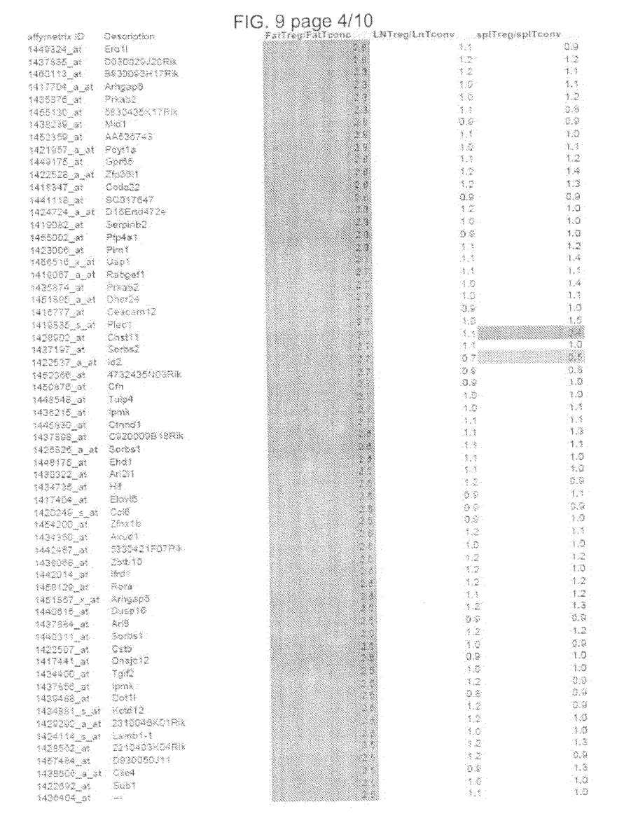

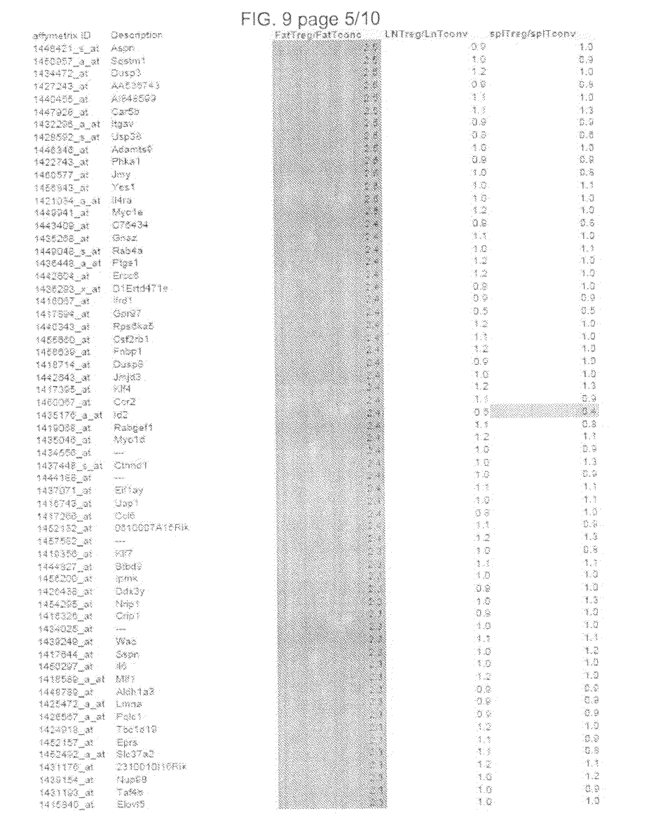

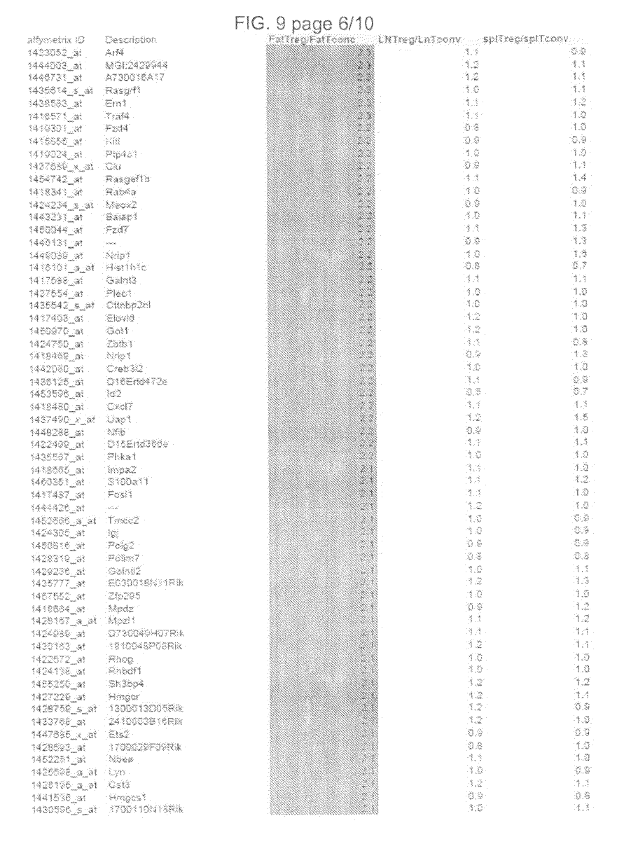

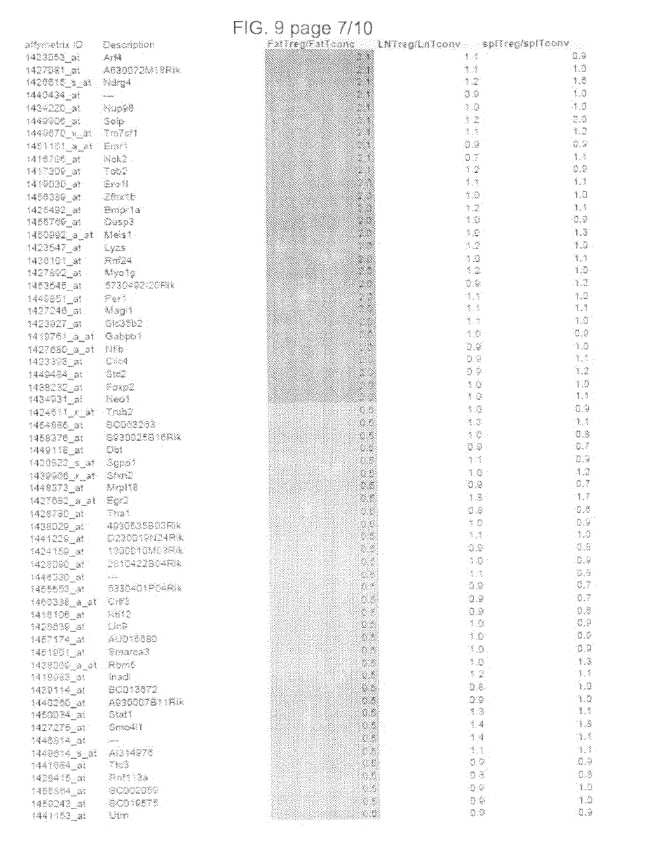

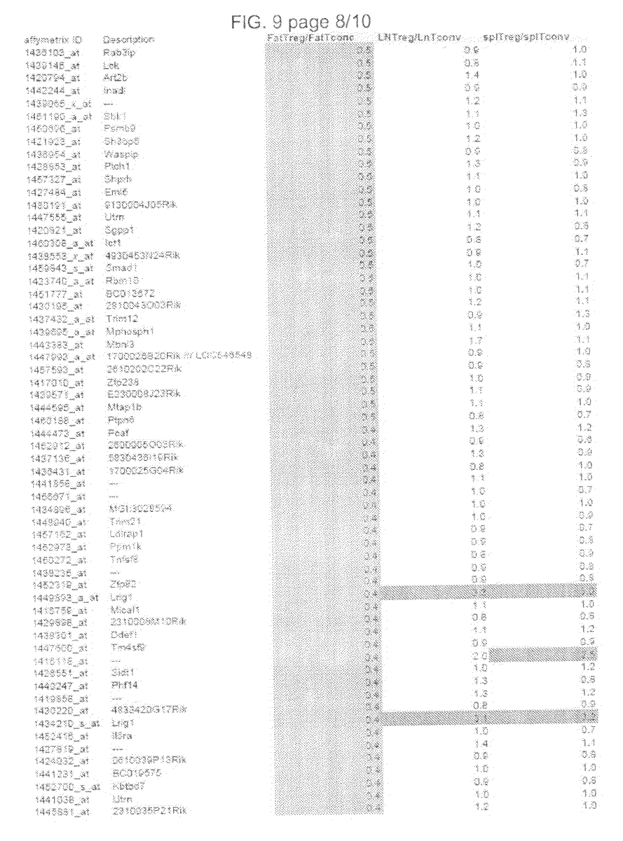

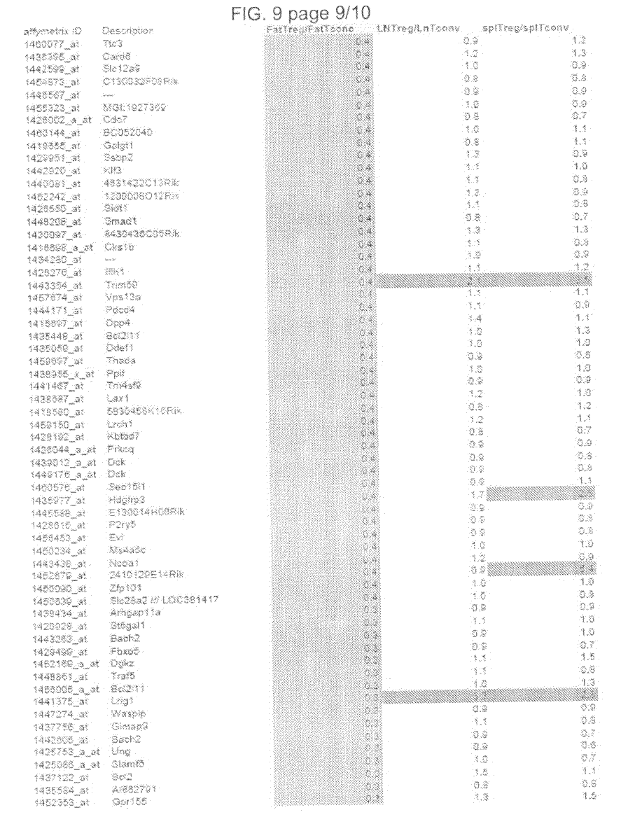

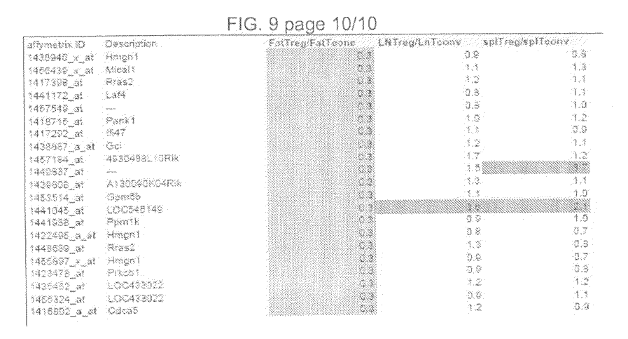

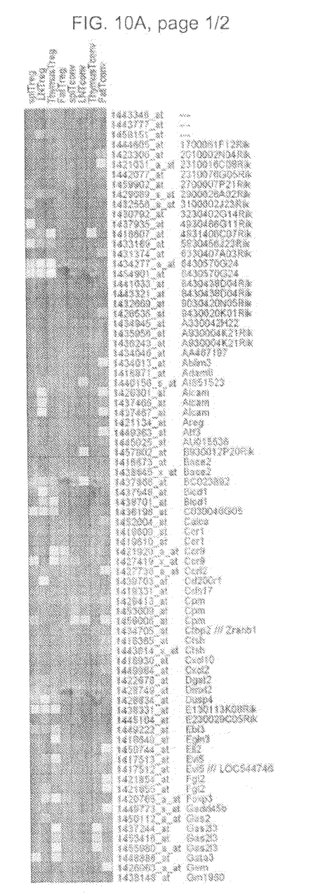

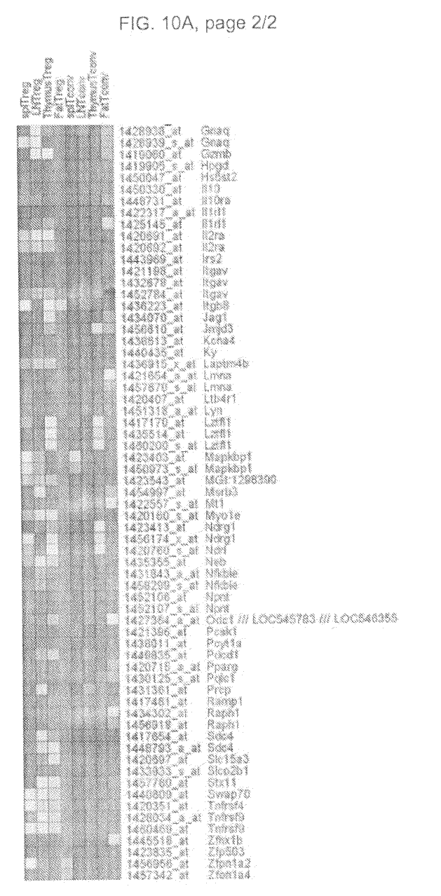

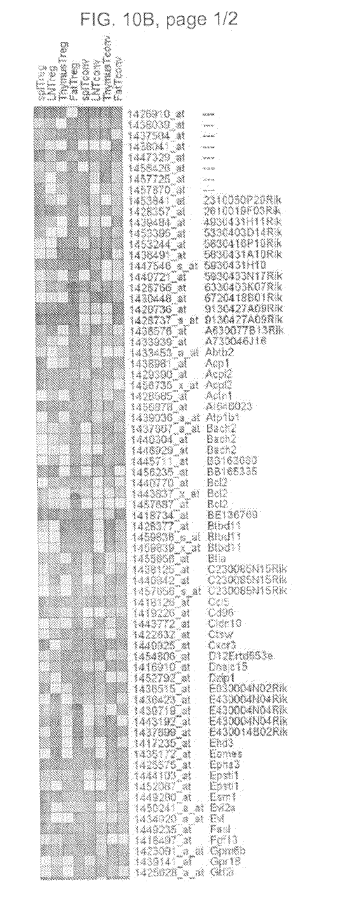

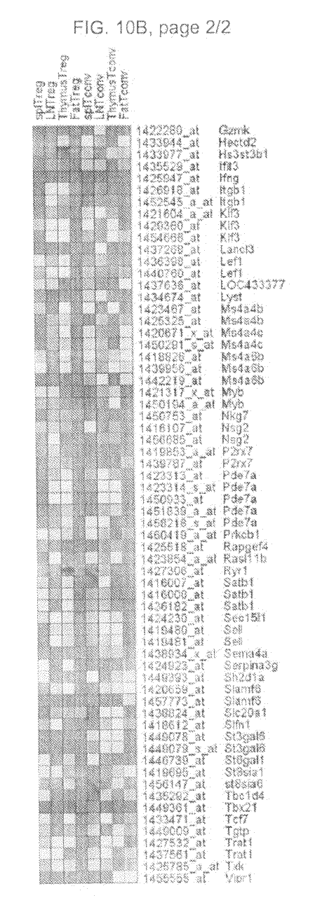

A large set of genes was over-expressed, many of them strikingly so, by the CD4⁺Foxp3⁺ T cells residing in abdominal adipose tissue, while not by the corresponding population at other sites examined (highlighted in light grey on FIG. 2, K and L; listed in FIG. 9). Chief amongst these were loci encoding molecules involved in leukocyte migration and extravasation: Gm1960 (an IL-10-inducible CXCR2 ligand (Samad et al., Mol Med 3, 37-48 (1997)), CCR1, CCR2, CCR9, CCL6, integrin alpha V, Alcam, CXCL2 and CXCL10 (FIG. 2K, FIGS. 9-10). On the other hand, some molecules of like function, eg CCL5 and CXCR3, were under-expressed in the visceral fat Tregs (FIG. 2K). Also remarkable were the extremely high IL-10 transcript levels in CD4⁺Foxp3⁺ abdominal adipose cells (FIG. 2, K vs L; FIG. 10). A 136-fold augmentation of IL-10 transcripts in fat vs LN Tregs was estimated from RT-PCR quantitation (FIG. 3C); the increase could also be detected by intracellular staining for IL-10 protein in the Tregs of fat versus spleen and lung (FIG. 3D). Interestingly, pathway analysis suggested that the Tregs not only produced large amounts of IL-10, but seemed also to be responding to it, as a number of genes downstream of the IL-10R were up-regulated in fat compared with in LN Tregs. While such an effect could also be discerned with fat Tconv cells, it was not as striking. Another set of genes was up-regulated specifically in CD4⁺Foxp3⁻ T cells residing in adipose tissue vis a vis their LN counterparts, but not in spleen versus LN (indicated as dark grey in FIG. 2, K and L; listed in FIG. 9). Some of these loci also coded for molecules implicated in migration and extravasation, including CXCR3 and CCL5. Fat Tconv cells appeared to be highly polarized to a TH1 phenotype as they expressed high levels of Tbet and IFN-γ transcripts (FIG. 2K, FIG. 3C, and FIG. 10), abundant intracellular interferon (IFN)-γ and tumor necrosis factor(TNF)-a (FIG. 3, D and E), and little if any intracellular IL-4 (FIG. 3D).

### Example 3

### T Cell Receptor (TCR) Repertoire

The T cell receptor (TCR) repertoire represents another parameter for assessing the degree of similarity of T cell populations: for example, it has been shown that Treg and Tconv cell populations have distinct repertoires, with only limited overlap (Wong et al., J Immunol 178, 7032-7041 (2007); Hsieh et al., Nat. Immunol 7, 401-410 (2006); Pacholczyk et al., Immunity 25, 249-259 (2006)). In addition, the TCR repertoire of Treg cells in the abdominal adipose tissue might give an indication of whether their abundance reflects an influx and/or retention of cells of a particular specificity or a local cytokine-induced conversion (Kretschmer et al., Nat. Immunol 6, 1219-1227 (2005)).

To render the repertoire analysis more manageable and interpretable, we exploited the Limited (LTD) mouse line, wherein TCR diversity is restricted to the complementary-determining region (CDR)₃ a via the combination of a transgenic TCRα minilocus and the TCRα-knockout mutation (Correia-Neves, C. Waltzinger, D. Mathis, C. Benoist, Immunity 14, 21-32 (2001)). CDR3α sequences were determined from 98 individually sorted visceral fat CD4⁺CD25⁺ cells that also expressed Foxp3 RNA, and their distribution was compared with that of CDR3α sequences from fat Tconv cells or LN Treg and Tconv cells. (Insufficient numbers of Treg cells were isolated from subcutaneous fat to perform a parallel TCR sequence analysis on this depot).

These experiments were performed as detailed in Wong et al., J Immunol 178, 7032-7041 (2007). Briefly, lymphocytes were first sorted in bulk as Vα2⁺Vβ5⁺CD4⁺CD8α⁻B 220⁻ and either CD25⁺ or CD25⁻, before resorting as individual cells into wells of 96-well PCR plates containing the RT reaction mix. The plates were incubated for 90 minutes at 37° C., then heat inactivated for 10 minutes at 70° C. Plates were replicated by transferring 5 μl of the cDNA into an empty plate. Nested PCR amplification was performed and contamination monitored in the replicates for Foxp3 or Vα2 as previously described (Correia-Neves, C. Waltzinger, D. Mathis, C. Benoist, Immunity 14, 21-32 (2001); Wong et al., J Immunol 178, 7032-7041 (2007)). Vα2 amplifications were prepared for automated sequencing Shrimp Alkaline Phosphatase (Amersham) and Exonuclease I (New England Biolabs) as previously detailed (Wong et al., J Immunol 178, 7032-7041 (2007)). Products were subjected to automated sequencing (Dana-Farber/Harvard Cancer Center High-Throughput Sequencing Core). Raw sequencing files were filtered for sequence quality, and processed in automated fashion.

As expected, the “heat maps” generated from these sequences (FIG. 4A) revealed distinct TCR repertoires for the LN Treg and Tconv populations, with only limited overlap. Similarly, the fat Treg and Tconv populations also had different repertoires, rendering it very unlikely that the accumulation of Foxp3⁺Treg cells in the abdominal adipose tissue resulted from local conversion of Tconv cells. Interestingly, the fat Tregs had a very restricted distribution of sequences, representing a distinct subset of those normally found in their LN Treg counterparts. The CDR3α sequences characteristic of fat Tregs were sometimes independently generated by different nucleotide sequences: 50% of sequences found more then three times per individual mouse (3/6) showed such nucleotide variation (FIG. 4A). In contrast, none of the fat Tconv cells (0/10) did (FIG. 4A), suggesting the repeated selection of Tregs with similar antigen receptors, rather than the proliferation of a single clone. The sequences were reproducibly frequent in different mice, again pointing to TCR-driven selection (FIG. 4A). These data indicate that the specificity of the TCR may be instrumental in generating the high frequency of Tregs in visceral fat, perhaps through local recognition of cognate antigen, reminiscent of recent findings that the repertoire of Tregs in peripheral lymphoid organs is enriched for autoreactive specificities (Hsieh et al., Immunity 21, 267-277 (2004)).

Indeed, fat Tregs displayed unusually high levels of the early activation markers CD69 and Ly6c (FIG. 4B), although it remains possible that such increases instead, or also, reflect cytokine influences. Though transforming growth factor (TGF)-β is readily detectable in adipose tissue (Samad et al., Mol Med 3, 37-48 (1997)), and it is known to promote Treg cell differentiation/survival (Chen et al., J Exp Med 198, 1875-1886 (2003); Peng et al., Proc Natl.Acad Sci U S.A. 101, 4572-4577 (2004); Marie et al., J Exp Med 201, 1061-1067 (2005)), its effects are an unlikely explanation for the high representation and activation state of Tregs in fat because the typical changes in gene expression promoted by this growth factor were not observed in this population. For example, CD103 was not up-regulated (FIG. 2, I and J, and FIG. 3B). This observation also argues against TGF-β-mediated conversion of CD4⁺Foxp3⁻ to CD4⁺Foxp3⁺ cells in visceral fat, as has been observed in a few systems (Kretschmer et al., Nat.Immunol 6, 1219-1227 (2005)).

### Example 4

### Treg Response to Adiposity in Models of Obesity

To learn how this peculiar population of Tregs responds to excess adiposity, it was examined in three mouse models of obesity: leptin-deficient mice (ob/ob) (Pelleymounter et al., Science 269, 540-543 (1995)), agouti heterozygotes (ag/wt) (Klebig et al., Proc Natl.Acad Sci U S.A. 92, 4728-4732 (1995)), and mice chronically fed a high-fat diet (HFD) (Cai et al., Nat. Med 11, 183-190 (2005)), all on the B6 genetic background and all displaying insulin resistance.

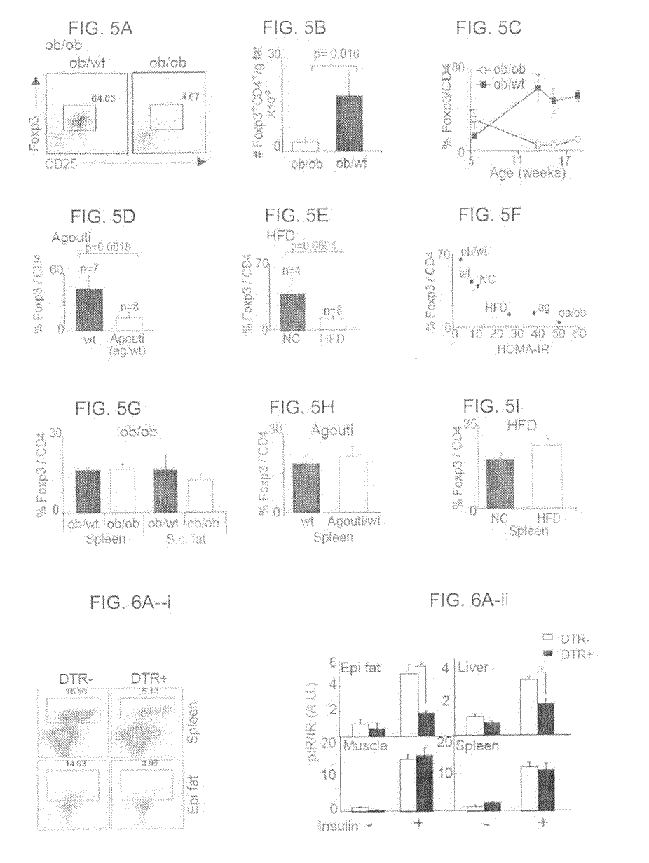

Strikingly, the Treg population in abdominal fat was drastically reduced in adult ob/ob mice, whether the fraction of Tregs in the CD4⁺ compartment or the number of Tregs per gram of fat was quantitated (FIGS. 5A and B). While five-week-old leptin-deficient animals had somewhat higher (p=0.02) levels of CD4⁺Foxp3⁺ T cells in visceral fat (30%) than did wild-type age-matched littermates (10%), this subset progressively declined in the former case and rose in the latter (FIG. 5C) (p=0.0011). The normal representation of Tregs in the spleen and subcutaneous fat of ob/ob mice (FIG. 5G) argue that the deficiency of this subset in visceral fat was not just a reflection of the leptin deficiency; indeed, the absence of leptin was recently reported to foster the proliferation of Tregs (De, V et al., Immunity 26, 241-255 (2007)). This point is underlined by the reduced levels of CD4⁺Foxp3⁺ cells in abdominal fat but not at other sites in the ag/wt mice and in HFD-fed mice (FIGS. 5D and E; H and I). The reductions were not as striking as for ob/ob animals, consistent with less insulin resistance in the latter two models. Indeed, we saw a good correlation between insulin resistance and the fraction of Tregs in abdominal fat (FIG. 5F).

### Example 5

### Treg Control of Adipose Cell Function—Effect of Depletion

The observed correlation between obesity and insulin resistance on the one hand and a dearth of CD4⁺Foxp3⁺ cells in abdominal adipose tissue on the other hand suggests that Tregs might be involved in controlling relationships between local and/or systemic metabolic and inflammatory parameters. To directly test the impact of Tregs on the local inflammatory status of adipose tissue and on local and systemic insulin resistance, loss-of-function experiments were performed.

Given that it was not currently feasible to ablate Tregs specifically in the fat, mice expressing the diphtheria toxin (DT) receptor (R) under the control of the Foxp3 transcriptional regulatory elements were employed, wherein administration of DT results in punctual systemic depletion of Tregs. DT has no adverse effects on the feeding behavior or weight of the mice. Also, the cell death induced by DT is apoptotic, and therefore does not set off a pro-inflammatory immune response (Bennett and Clausen, Trends Immunol 28, 525-531 (2007); Thorburn et al., Clin Cancer Res. 9, 861-865 (2003); Miyake et al., J Immunol 178, 5001-5009 (2007); Bennett et al., J Cell Biol 169, 569-576 (2005)), prompting wide-spread use of this approach to probe diverse immunological issues through specific ablation of particular cell-types, including Tregs (Bennett and Clausen, Trends Immunol 28, 525-531 (2007); Duffield et al., Am J. Pathol. 167, 1207-1219 (2005); Duffield et al., J Clin Invest 115, 56-65 (2005); Walzer et al., Proc Natl.Acad Sci U S.A. 104, 3384-3389 (2007)). Because Treg-deficient mice develop multi-organ autoimmunity beyond 2 weeks post-depletion (Kim et al., Nat.Immunol 8, 191-197 (2007)), this strategy required evaluation of early indicators of potential Treg function, namely alterations in adipose tissue mRNAs encoding inflammatory mediators or upstream changes in metabolic signaling pathways; previous data suggested that two weeks may be too early to see changes in many metabolic parameters, including performance in glucose-tolerance tests (GTTs) (Yuan et al., Science 293, 1673-1677 (2001)). A line of NOD BAC transgenic mice expressing a diphtheria toxin (DT) receptor (R)-eGFP fusion protein under the dictates of Foxp3 transcriptional regulatory elements was generated. In brief, the BAC span from 150 kb upstream to 70 kb downstream of Foxp3 transcription start site was used. DTR-eGFP cDNA with stop codon was inserted between the first and second codon of the Foxp3 open reading frame. Recombinant Foxp3DTR BAC was directly injected into NOD mice.

Routinely, 85-90% of Tregs were eliminated in the spleen and LNs two days after DT administration to these animals, similar to what has been described by the Rudensky group(Kim et al., Nat. Immunol 8, 191-197 (2007)).

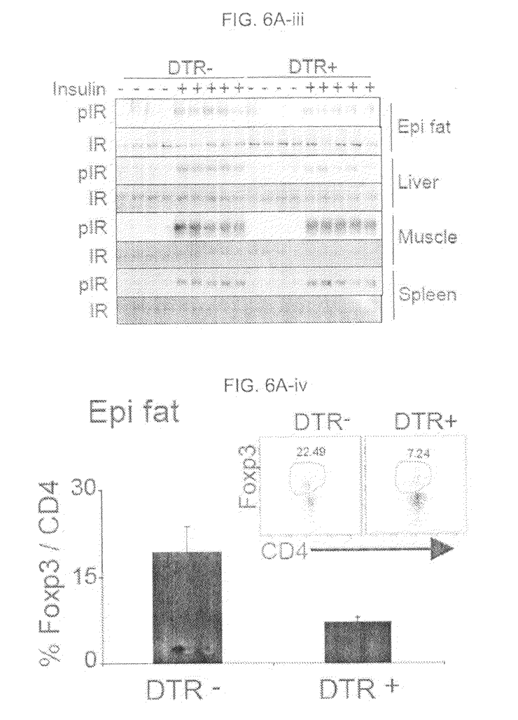

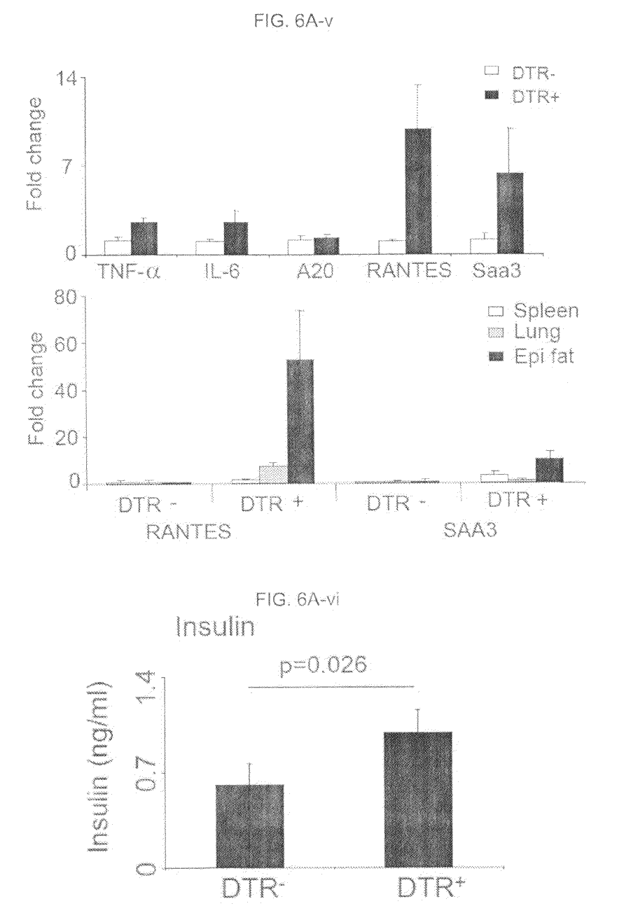

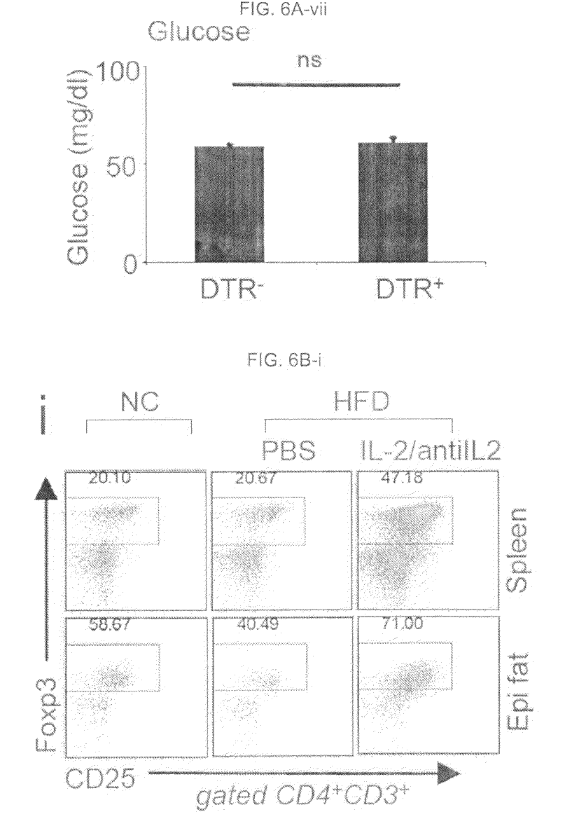

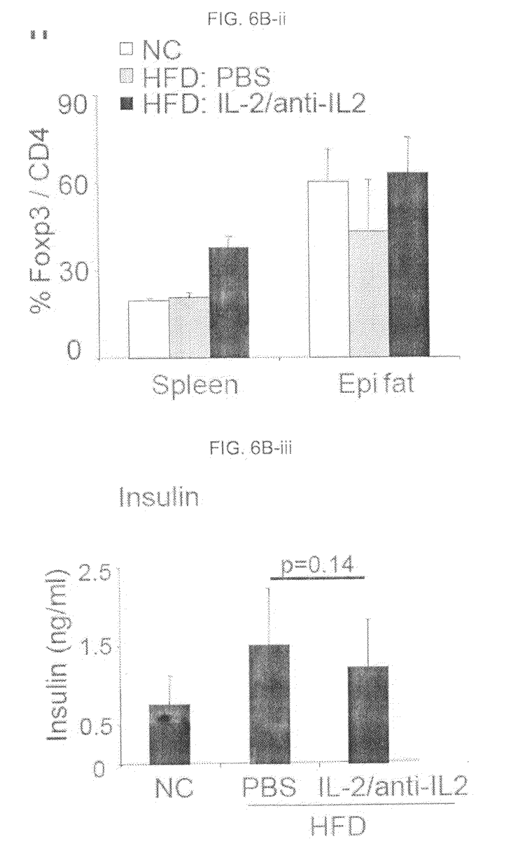

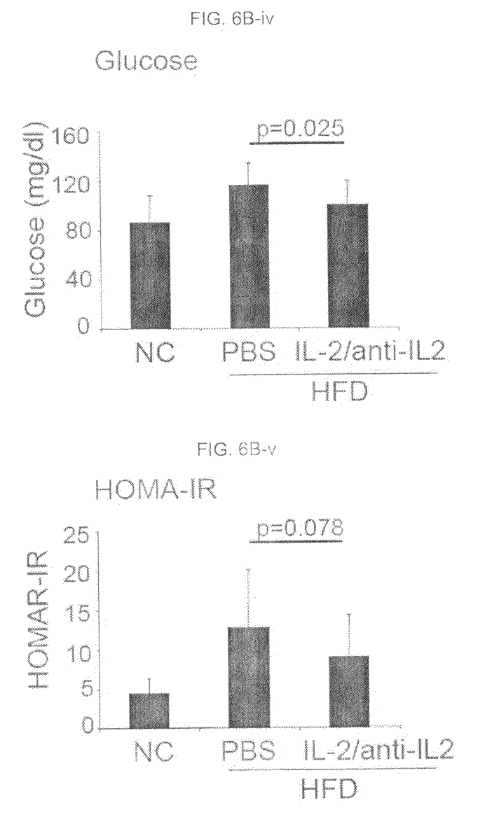

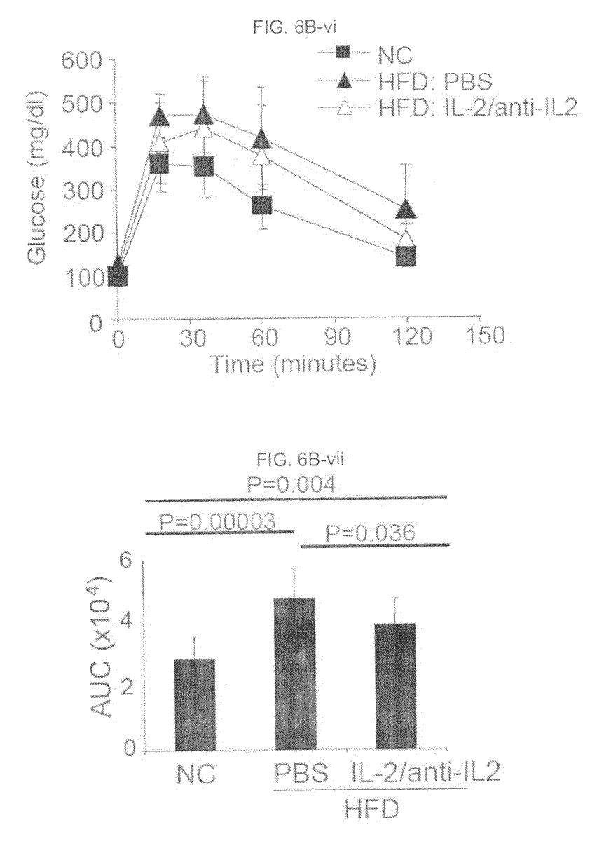

For one set of experiments, 10-week-old male mice were treated with DT every other day for four days, which reduced the Treg representation in abdominal fat to about ¼ the normal (FIG. 6A-i, bottom panels), while the spleen and lung populations were at about ⅓ the usual (FIG. 6A-i, top panels and additional data not shown). The depletion of Tregs was accompanied by substantial decreases in insulin-stimulated insulin-receptor (IR) tyrosine phosphorylation in epidydimal fat and liver, but not muscle and spleen (FIGS. 6A-i and 6A-iii). Parallel results were obtained on AKT phosphorylation. At this early time-point, in vivo metabolic changes were marginal, so we conducted a second set of experiments in which mice were treated with DT for longer times.

Mice injected with DT every other day for 9 days had a Treg fraction of about 30% the usual in the fat, while the spleen, lung and LN populations had bounced back to about 70% the normal (6A-iv). Concomitantly, many of the genes encoding inflammatory mediators (e.g., tumor necrosis factor (TNF)-α, IL-6, A20, RANTES, Serum Amyloid A (SAA)-3 were induced in the visceral fat depot (FIG. 6A-v, upper panel), and much less so in the spleen and lung (FIG. 6A-v, lower panel). Insulin levels were elevated in the Treg-depleted mice, demonstrating insulin resistance (FIG. 6A-vi), although fasting blood-glucose levels at this early time-point were unchanged, consistent with adequate 13-cell compensation (FIG. 6A-vii).

### Example 6

### Treg Control of Adipose Cell Function—Effect of Expansion

As concerns gain-of-function approaches, the lability and low recoverable numbers of visceral fat Tregs rendered unsuccessful our many attempts at standard transfer experiments; transfer of more limited numbers of fat Tregs into lymphodeficient recipients also proved problematic because the resultant homeostatic proliferation altered the phenotype of the transferred population, perhaps most relevantly its profile of cell-surface homing receptors (data not shown). Therefore, as an alternative means to achieve gain-of-function, in situ expansion of Tregs was achieved, via injection of a particular recombinant IL-2:anti-IL-2 monoclonal antibody (mAb) complex demonstrated by Sprent and collaborators to selectively grow Treg cells (Boyman et al., Expert Opin Biol Ther. 2006 December; 6(12):1323-31), and subsequently employed by multiple groups to this end (e.g., Tang et al., Immunity 28, 687-697 (2008)).

In these experiments, mice were purchased from Jackson Laboratory (Bar Harbor Me.) that had been fed for 12 weeks with HFD in the Jackson facility. Complexes of the anti-IL-2 mAb JES6-5H4 (BD Pharmingen) and mouse IL-2 (PeproTech) were prepared and i.p.-injected as described (Boyman et al., Science 311, 1924-1927 (2006)). In brief, 30 ug of anti-IL2 and 1 ug mIL-2 per mouse were incubated for 20 minutes on ice followed by i.p. injection. Mice received daily injections for 6 days and were analyzed on day 14; Control mice were injected with saline (PBS). In some experiments mice were fed with HFD for 8 weeks (60 kcal % fat from Research Diet (New Brunswick, N.J.; Cat# D12492)), and were injected with the complex for 9 days.

Daily injections of the complex for 6 days into mice pre-fed an HFD for fifteen weeks did substantially increase the fraction of Tregs in the spleen and in abdominal fat vis a vis PBS-injected controls (37+/−4% vs. 21+/−2% for spleen and 63+/−12% vs. 43+/−17% for abdominal fat (FIG. 6B-, i and ii). Since the complex-injected mice had been pre-challenged with an FWD, we could assess the influence of an elevated representation of Tregs on various indicators of insulin resistance. Blood-glucose levels were significantly lower in the HFD-fed mice with more Tregs (FIG. 6B-iv). While blood-insulin levels (FIG. 6B-iii), HOMA-IR (FIG. 6B-v) and glucose tolerance (measured via a GTT) (FIG. 6B-vi) all trended towards lower values in the Treg-enriched HFD-fed animals, these differences fell short of statistical significance, probably due to the greater experimental variability inherent in these assays. Small differences are also not surprising given the short experimental window (Yuan et al., Science 293, 1673-1677 (2001)). In order to enhance power, a number of additional HFD-fed mice were injected with IL-2:anti-IL-2 complexes vs PBS under similar conditions, accumulating a total of 11 mice per each group. The Treg fraction in the complex-injected HFD-fed mice ranged from 40-83% (Av=68=/−13%). As indicated in FIG. 6B-vii, both HFD-fed groups were glucose intolerant vis a vis control mice fed normal chow (NC); however the complex-injected group, with the highest levels of Tregs, showed a significant improvement compared with the PBS-injected group.

These findings indicate that Tregs guard against excessive inflammation of the adipose tissue and local and downstream systemic consequences, and strongly suggest that Tregs residing in the fat are responsible.

### Example 7

### Treg Control of Adipose Cell Function—Effect of Expansion

A likely mechanism by which T cells residing in adipose tissue impact neighboring cells is through soluble mediators. Thus, the influence of the major cytokines differentially produced by Treg and Tconv cells was explored in fat vis a vis at other sites: according to our gene-expression profiling, these cytokines were IL-10 and IFN-γ, respectively.

3T3-L1 cells obtained from ATCC (Manassas, Va.) were cultured and induced to differentiate into adipocytes as previously described (Frantz et al., J Biol. Chem. 272, 2659-2667 (1997)). Once fully differentiated, the cells were treated with IL-10 (PeproTech, Rocky Hill, N.J.) for 24 hours and then with TNF-α 1 ng/ml for an additional 24 hours. In some experiments cells were treated for 24 hours with IFN-γ 10 ng/ml or IL-1β 10 ng/ml (PeproTech). The cells were harvested and mRNA extracted with Trizol (Invitrogen-Gibco). cDNA was prepared by using the Advantage RT-PCR kit (Clontech, Mountain View, Calif.) as recommended, and gene-expression levels were analyzed using the ABI prism 7000 machine (Applied Biosystems, Foster city, CA) and either ABI prism Taqman™ or Sybr™ green master mixes. Transcription levels were normalized to 18S and 36B4 expression (equivalent results).

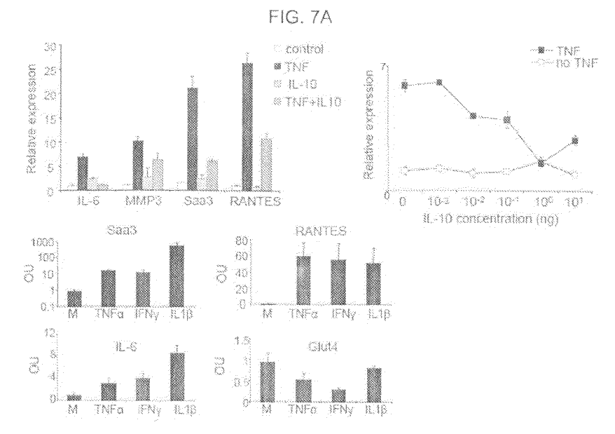

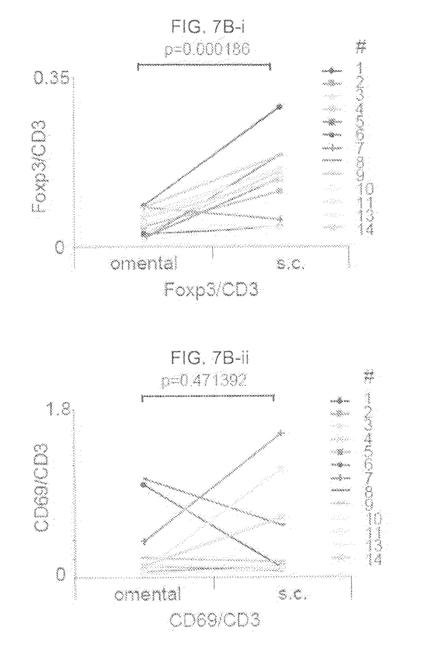

Fully differentiated, lipid-laden 3T3-L1 adipocytes were pretreated or not for 48 h with IL-10, and were subsequently stimulated for 24 h with TNF-α, an established method for in vitro induction of insulin resistance (FIG. 7A, left and center). TNF-α induced changes in adipocyte expression of a number of transcripts encoding inflammatory mediators, for example IL-6, RANTES, SAA-3 and matrix metalloproteinase (MMP)3. Strikingly, IL-10 inhibited the TNF-α-induced expression of all of these mRNAs. TNF-α has also been shown to down-modulate insulin-dependent tyrosine phosphorylation of insulin receptor substrate (IRS)1 and to inhibit Glut4-mediated glucose uptake in 3T3-L1 adipocytes, and these effects, too, were reversed by IL-10 (Lumeng et al., J Clin Invest 117, 175-184 (2007)), indicating that this cytokine reverts insulin resistance by a mechanism directly impinging on adipose tissue cells (i.e., is cell autonomous). In striking contrast to the anti-inflammatory effects of this mediator made by visceral fat Tregs, a major product of the Tconv cells at this site, IFN-γ, was pro-inflammatory in the same in vitro assay system: expression of SAA3, RANTES and IL-6 transcripts were all induced, and Glut4 mRNA was down-regulated (FIG. 7A iii).

### Example 8

### Tregs in Human Adipose Tissues

To evaluate the applicability of these findings to human treatment, a set of paired snap frozen omental and subcutaneous fat tissues from a number of individuals with an average body:mass index (BMI) of 44.85, thus falling within the obese (30-39.9) and morbidly obese (>40) range) was obtained, and quantitative PCR was performed for FOXP3, CD3 and CD69. Given that the samples were frozen, flow cytometric analysis on or purification of lymphocyte populations were not possible, but FOXP3 transcript levels were evaluated by PCR (FIG. 7Bi). FOXP3 mRNA was readily detectable in both fat depots. Consistent with the observations on obese mice, there were higher levels of FOXP3 transcripts, presumably an indicator of Treg cells, in the subcutaneous adipose tissue. This result was not simply an artifact of more activated Tconv cells at that location, a potential issue given that in humans activated T cells also express FOXP3 (Walker et al., J Clin Invest 112, 1437-1443 (2003)), because there was no parallel increase in the mRNA encoding the early activation marker CD69 (FIG. 7Bii). These data suggest that the findings described herein are translatable to humans.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of inhibiting, delaying, or reducing the development or severity of obesity-associated disorders in a subject, the method comprising:
obtaining a population of fat-tissue specific regulatory T cells produced by the method of claim 2; and administering said population of fat-tissue specific regulatory T cells to the subject.

2. A method of producing a population of fat-tissue specific regulatory T cells, the method comprising:
obtaining an initial population of Foxp3+CD25+CD4+ regulatory T cells;
culturing said initial population of T cells, and
selecting cells from the cultured population of T cells that express one or more of IL-10, Gm1960, CCR1, CCR2, CCR9, CCL6, CXCL5, CXCL7, CXCL10, CXCL2, integrin alpha V, and Alcam, thereby forming a population of fat-tissue specific regulatory T cells.

3. The method of claim 2, further comprising:
engineering said initial population of T cells to express IL-10, and culturing said cells, optionally in the presence of adiponectin.

4. The method of claim 2, wherein the initial population of cells comprises regulatory T cells from peripheral blood.

5. The method of claim 2, wherein the initial population of cells comprises regulatory T cells from fat tissue.

6. The method of claim 1, wherein said population of fat-tissue specific regulatory T cells is administered systemically.

7. The method of claim 1, wherein said population of fat-tissue specific regulatory T cells is administered locally to a fat tissue.

8. The method of claim 2, comprising selecting cells from the cultured population of cells that express all of Gm1960, CCR1, CCR2, CCR9, CCL6, CXCL5, CXCL7, CXCL10, CXCL2, integrin alpha V, and Alcam.

9. The method of claim 2, wherein the initial population of T cells is cultured in the presence of one or both of interleukin 2 (IL-2) and transforming growth factor beta (TGFβ).

10. The method of claim 2, wherein the initial population of cells is cultured in the presence of an anti CD3 antibody, and optionally a costimulatory molecule.

11. The method of claims 2, wherein the cells are genetically engineered to express Fat Treg-specific T-Cell Receptors (TCRs).

12. A population of cells produced by the method of claim 2.

13. A method of treating obesity or obesity-associated conditions, or both, in a subject, the method comprising administering a therapeutically effective amount of interleukin (IL)-10 and optionally adiponectin to the subject.

14. The method of claim 13, wherein IL-10 and adiponectin are administered systemically.

15. The method of claim 13, wherein IL-10 and adiponectin are administered locally to a fat tissue of the subject.

16. The method of claim 13, wherein IL-10 and adiponectin are administered in a single composition.

17. A pharmaceutical composition comprising IL-10 and adiponectin as active ingredients, and a physiologically acceptable carrier.

18. A method of treating obesity or obesity-associated conditions or both in a subject, the method comprising selecting a subject based on a diagnosis of obesity, and administering a therapeutically effective amount of a composition comprising an interleukin (IL)-2:anti-IL-2 monoclonal antibody (mAb) complex.

19. The method of claim 1, wherein the obesity-associated disorder is insulin resistance.

20. The method of claim 1, wherein the subject does not have an autoimmune disorder.

21. The method of claim 1, further comprising selecting a subject that does not have an autoimmune disorder.

22. The method of claim 1, wherein the subject does not have type 1 diabetes.

23. The method of claim 13 wherein the obesity-associated condition is insulin resistance.

24. The method of claim 18, wherein the obesity-associated condition is insulin resistance.

25. The method of claim 1, wherein the fat-tissue specific regulatory T cells are autologous.

26. The method of claim 1, wherein the fat-tissue specific regulatory T cells are not autologous.
